SEQUENCE LISTING (4 sequences)

SEQ ID NO: 1 - DNA, 125 bp, Artificial sequence
  Synthetic
ggaagcgaaaatgaaattgactgggactttccggaggaaaaactgtttcatacagaaggcgtggatgtccatattaggatgagtcagtgacgtcagagcctgatttccccgaaatgatgagctag

SEQ ID NO: 2 - DNA, 3135 bp, Artificial sequence
  betaKlotho CDS - codon optimized
atgaagcctggatgtgccgccggaagccccggcaacgagtggatcttcttcagcaccgacgagatcaccacccggtacagaaacaccatgagcaacggcggcctgcagcggagcgtgatcctgtctgctctgatcctgctgagagccgtgaccggctttagcggcgacggcagagccatctggtccaagaaccccaacttcacccccgtgaacgagagccagctgttcctgtacgataccttccccaagaacttcttctggggcatcggcacaggcgccctgcaggtggaaggcagctggaagaaggatggcaagggccccagcatctgggaccacttcatccacacccacctgaagaacgtgtccagcaccaacggcagcagcgacagctacatcttcctggaaaaggacctgagcgccctggacttcatcggcgtgtccttctaccagttcagcatcagctggcccagactgttccccgacggcatcgtgacagtggccaatgccaagggcctgcagtactacagcaccctgctggacgccctggtgctgagaaacatcgagcccatcgtgaccctgtaccactgggacctgccactggctctgcaggagaaatacggcggctggaaaaacgacaccatcatcgacatcttcaacgactacgccacctactgcttccagatgttcggcgacagagtgaagtactggatcaccatccacaacccctacctggtggcctggcacggctatggcaccggaatgcatgcccctggcgagaagggaaatctggccgccgtgtacaccgtgggccacaacctgatcaaggcccacagcaaagtgtggcacaactacaacacccacttccggccccaccagaagggctggctgtctatcacactgggcagccactggatcgagcctaaccgcagcgagaacaccatggatatcttcaagtgccagcagagcatggtgtccgtgctgggatggttcgccaaccccattcatggcgacggcgactaccccgagggcatgcggaagaaactgttcagcgtgctgcccatcttcagcgaggccgagaagcacgagatgcggggcaccgccgatttcttcgcctttagcttcggccccaacaacttcaagcccctgaataccatggccaagatgggccagaatgtgtccctgaacctgagagaggccctgaactggatcaagctggagtacaacaacccccggatcctgatcgccgagaacggctggttcaccgacagcagagtgaaaaccgaggacaccaccgccatctatatgatgaagaacttcctgagccaggtgctgcaggctatccggctggatgagatccgggtgttcggctacacagcctggtcactgctggatggcttcgagtggcaggacgcctacaccatcagacggggcctgttctacgtggacttcaacagcaagcagaaagagcggaagcccaagagcagcgcccactactacaagcagatcatcagagagaatggcttcagcctgaaagaaagcacccccgacgtgcagggccagttcccctgtgatttttcctggggcgtgaccgagagcgtgctgaagcctgagtctgtggcctccagcccccagtttagcgacccccacctgtacgtgtggaacgccaccggcaaccggctgctgcatagagtggaaggcgtgcggctgaaaaccagacccgcccagtgcaccgacttcgtgaacatcaagaaacagctggaaatgctggcccggatgaaagtgacccactacagattcgccctggattgggcctccgtgctgcctaccggaaacctgtctgccgtgaacagacaggccctgaggtactacagatgcgtggtgtccgagggcctgaagctgggcatcagcgccatggtcacactgtactaccccacccacgcccacctgggactgcctgaacctctgctgcatgccgacggctggctgaatccttctacagccgaggccttccaggcctacgccggactgtgctttcaggaactgggcgacctcgtgaagctgtggatcacaatcaacgagcccaacagactgagcgacatctacaacagaagcggcaacgacacctacggcgctgcccacaatctgctggtggctcatgccctggcctggcggctgtacgacagacagttcagaccaagccagcggggagccgtgtctctgtctctgcatgctgattgggccgagcccgccaacccttacgccgactctcattggagagccgccgagcggttcctgcagttcgagatcgcttggtttgccgagcccctgttcaagaccggcgattaccctgccgccatgagagagtatatcgccagcaagcacagaagaggcctgagcagctccgccctgcctagactgacagaggccgaaaggcggctgctgaagggcaccgtggatttctgtgccctgaatcacttcaccaccagattcgtgatgcacgagcagctggccggcagcagatacgacagcgaccgggacatccagtttctgcaggacatcacccggctgagcagccctacaagactggccgtgatcccttggggcgtgcggaagctgctgagatgggtgcgcagaaactacggcgacatggacatctatatcaccgccagcggcatcgacgaccaggccctggaagatgaccggctgcggaagtactacctgggcaagtacctgcaggaagtgctgaaggcctacctgatcgacaaagtgcggatcaagggctactacgccttcaagctggccgaggaaaagagcaagcccagattcggcttcttcaccagcgacttcaaggccaagtccagcatccagttctacaacaaagtgatcagcagcaggggcttcccattcgagaacagcagcagcagatgcagccagacacaggaaaacaccgagtgtaccgtgtgcctgttcctggtgcagaagaagcctctgatctttctgggctgctgcttctttagcacactggtgctgctgctgagcattgccatctttcagcggcagaagcggagaaagttttggaaggccaagaatctgcagcacatccccctgaaaaagggcaagcgggtggtgtcttga

SEQ ID NO: 3 - DNA, 60 bp, Artificial sequence
  Synthetic
ctcgggaaagggaaaccgaaactgaagcccctcgggaaagggaaaccgaaactgaagccc

SEQ ID NO: 4 - DNA, 70 bp, Artificial sequence
  Synthetic
tggggactttccgctggggactttccgctggggactttccgctggggactttccgctggggactttccgc

(19) United States
(12) Patent Application Publication
(10) Pub. No.: US 2021/0293784 A1
(43) Pub. Date: Sep. 23, 2021

(54) SYSTEM FOR THE ADAPTATION OF CELL-BASED ASSAYS FOR ANALYSIS ON AUTOMATED IMMUNO-ASSAY PLATFORMS

(71) Applicant: SVAR LIFE SCIENCE AB, Malmõ (SE)

(72) Inventors: Michael TOVEY, PARIS (FR); Christophe LALLEMAND, PARIS (FR)

(21) Appl. No.: 16/500,341

(22) PCT Filed: Apr. 6, 2018
(86) PCT No.: PCT/EP2018/058890

§ 371 Date: Oct. 2, 2019

(30) Foreign Application Priority Data
Apr. 7, 2017 (EP) 17165502.0

(51) Int. Cl. G01N 33/50; C07K 14/735; C12N 15/86; C07K 14/47

(52) U.S. Cl. CPC G01N 33/5008; C07K 14/70535; C12N 2750/14143; C07K 14/4705; C12N 15/86

(57) ABSTRACT

Cell based assays are required for the detection of biological activity and hence are required for use as potency assays, detection of neutralizing antibodies and detection and quantification of the effector cell function of therapeutic antibodies or the quantification of the potency or neutralizing antibody response to virus vectors such as AAV vectors used in gene therapy. Cell-based assay are also required for the quantification of antibody mediated effector functions including complement-dependent cytotoxicity (CDC), antibody-dependent cellular phagocytosis (ADCP), and antibody-dependent cell-mediated cytotoxicity (ADCC). Cell based assays are difficult to adapt for use on automated immuno-assay platforms. Reporter-genes that encode readily visible proteins that can be quantified using fluorescence or luminescence such as luciferases (firefly, Renilla, Gaussia, Nano Luciferase etc), fluorescent proteins such as green fluorescent protein (GFP) or dsRED or an enzyme such as chloramphenicol acetyltransferase (CAT) or a protease and respond to signal transduction, that is directly related to the mechanism of action of a drug, can be used to quantify the potency of a drug following binding of a drug to a soluble target or cell surface target (receptor or other cell-surface molecule), anti-drug neutralizing antibodies, and therapeutic antibody induced effector cell function. The principal of the invention is that reporter-gene product or by-product produced either during the course of the cell-based assay or onconclusion of the cell based assay is quantified either in the cell medium or cell supernatant, for a secreted protein, or following lysis of the cells with a suitable passive lysis buffer. The reporter-gene product such as firefly luciferase is then detected in the cell medium or cell supernatant or cell lysate using an antibody pair (monoclonal or polyclonal) specific for the gene product such as a firefly luciferase labelled with the dual detection system specific for an ELISA or a particular automated assay platform such as Meso Scale Discovery electro-chemiluminescence (MSD-ECL), Luminex, SMC, Alpco, AlphaLISA, Gyros or label free detection using SPR such as the Biacore platform. The expression of the reporter-gene product such as firefly luciferase can be normalized with respect to the expression of a second reporter gene product such as Renilla luciferase or Nano Luciferase under the control of a constitutive promoter.

### CROSS-REFERENCE TO RELATED APPLICATIONS

This application is a national phase application under 35 U.S.C. § 371 of PCT International Application No. PCT/EP2018/058890, filed Apr. 6, 2018, which claims priority to European Application No. 17165502.0, filed Apr. 7, 2017, which are hereby incorporated by reference in its entirety.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 5,432 Byte ASCII (Text) file named “38525-251_ST25.TXT,” created on Mar. 10, 2021.

### FIELD OF THE INVENTION

The present invention relates to a novel system and its use in methods for determining the potency, neutralizing antibody response, and effector cell function of a therapeutic antibody using an automated immuno-assay platform. The system according to the invention may be used in a kit or a kit of parts that may be used in a diagnostic context. Importantly, the system according to the invention may be used to determine the effectiveness of a treatment based on the use of a therapeutic protein, recombinant or naturally occurring, or a therapeutic antibody, or the transgene expressed by a virus vector such as an Adeno Associated Virus (AAV) vector, or the immune response elicited by the therapeutic protein or therapeutic antibody, recombinant virus, or cells used in adoptive therapy, that may be advantageous as in the case of the induction of effector cell function or disadvantageous as in the case of a break in immune tolerance and the production of anti-drug antibodies.

### BACKGROUND OF THE INVENTION

Repeated administration of recombinant biopharmaceuticals can lead to a break in immune tolerance (1) and the production of anti-drug antibodies (ADA). In addition to adversely affecting pharmacokinetics, pharmacodynamics, bioavailability, and efficacy, ADAs can also cause immune complex disease, allergic reactions, and in some cases severe autoimmune reactions. Certain types of ADAs may also neutralize the activity of the protein therapeutic. Neutralizing antibodies (NAbs) block the biological activity of a biopharmaceutical either by binding directly to an epitope within or close to the active site of the protein or by binding to an epitope that prevents binding of the drug to a cell surface receptor. Development of neutralizing anti-drug antibodies is of particular concern in the treatment of chronic diseases, including certain forms of cancer and autoimmune or inflammatory diseases such as multiple sclerosis or rheumatoid arthritis. ADAs can result in the failure of the patient to respond to therapy and may even prove to be life threatening in the case of NAbs that cross-react with essential non-redundant endogenous proteins such as EPO or thrombopoietin (2,3). Drug-induced immunoglobulin IgE antibodies can also cause serious anaphylactic reactions (4). ADAs can also persist for long periods after cessation of treatment, thereby limiting subsequent treatment with the same drug (5). Assessment of immunogenicity is therefore an important component of drug safety evaluation in both preclinical and clinical studies and is a prerequisite for the development of less immunogenic and safer biopharmaceuticals.

Regulatory authorities recommend the use of a tiered approach for the assessment of immunogenicity consisting of an initial screening immuno-assay followed by the testing of positive samples in a confirmatory orthogonal immuno-assay (6, 7). Confirmed positive samples are then assayed for the presence of neutralizing antibodies. Regulatory authorities recommend the use of a cell-based assay, which reflects as closely as possible the mode of action of the drug under study, to detect and quantify neutralizing anti-drug antibodies. Immunogenicity studies often require the testing of hundreds or thousands of individual samples necessitating the use of an automated immuno-assay platform technology. Although immuno-assays such as bridging ELISA based screening or confirmatory assays can be readily adapted to run on automated assay platforms such as Meso Scale Discovery electro-chemiluminescence (MSD-ECL), Luminex, SMC, Alpco, AlphaLISA or Gyros, or label-free detection systems such as SPR systems including Biacore, cell-based assays are less amenable to adaptation to such platform technologies. Similarly, the activity of numerous therapeutic antibodies is mediated in part by immune-mediated effector cell function following binding of the variable regions of the antibody to a specific antigen on the surface of the target cell and the interaction of the Fc moiety of the antibody with a Fc receptor on an immune effector cell. Antibody mediated effector functions (8, 9) include complement-dependent cytotoxicity (CDC), antibody-dependent cellular phagocytosis (ADCP), and antibody-dependent cell-mediated cytotoxicity (ADCC). Assessment of antibody mediated immune effector functions require the use of cell-based assays that are difficult to adapt to analysis on an automated immuno-assay platforms.

We describe herein an invention that permits reporter-gene assays, including luciferase reporter-gene assays, for the quantification of the potency of biopharmaceuticals, the detection of neutralizing anti-drug antibodies or the quantification of immune mediated effector cell function such as CDC, ADCC, or ADCP to be quantifies using an ELISA, or on automated assay platforms such as the Gyros, MSD, or AlphaLISA systems, or by label-free detection using SPR such as the Biacore system, without the need to re-engineer either the reporter-gene cell lines and/or the instrument.

Consequently, present invention relates to use of a cell line according to the invention in an automated assay, such as e.g. an automated immunoassay.

Moreover, present invention also relates to use of a kit according to the invention in an automated assay, such as e.g. an automated immunoassay.

As mentioned above, the use does not require any modification or adaptation of the cell line or kit in order to be able to be used in an automated assay, such as e.g. an automated immunoassay.

### FIGURES

FIG. 1 Illustrates a bridging ELISA for the detection of firefly luciferase using a pair of anti-luciferase antibodies on the left-hand side (LHS) of the Figure and the iLite® NAb assay on the upper right-hand side (RHS) of the Figure and on the lower RHS of the Figure the luciferase detection system based on the use of a specific anti-luciferase antibody pair consisting of one molecule labelled with biotin bound to a streptavidin coated assay plate and a second anti-luciferase antibody molecule labelled with horseradish peroxidase (HRP) that allows biological activity detected by the cell-based iLite® NAb assay to be quantified using a bridging ELISA following addition of the HRP substrate and quantification of optical density using a spectrophotometer.

FIG. 2A illustrates the characteristics of the panel of anti-luciferase antibodies tested.

FIG. 2B illustrates the performance of the anti-luciferase antibody Ab-185924 (AbCam, UK) used as the capture antibody and the HRP labelled anti-luciferase antibody Ab-635 AbCam, UK) used as the detection antibody in a bridging ELISA.

FIG. 3A illustrates the results of a bridging ELISA for the detection of recombinant firefly luciferase comparing the relative efficacy of an in-house blocking buffer with that of 2 commercially available blocking buffers, incubated at 25° C. for 2 hours, for the detection of recombinant firefly luciferase using a pair of anti-luciferase antibodies (coating antibody AbCam UK, Catalogue No Ab-64564; detection antibody AbCam UK, Catalogue No Ab-635) prior to the addition of the HRP substrate and quantification of optical density at 450 nm using a spectrophotometer. FIG. 3B illustrates the table with measured values accompanying the graph in FIG. 3A.

FIG. 4A illustrates the results of a bridging ELISA comparing the relative efficacy of 3,3′,5,5′-tetramethylbenzidine (TMB) and luminol for the detection of firefly luciferase in extracts of FGF-21 responsive cells treated with increasing concentrations of bFGF (coating antibody AbCam UK, Catalogue No Ab-222862; detection antibody AbCam UK, Catalogue No Ab-635), compared with the detection of firefly luciferase activity using One-Glo substrate (Promega). FIG. 4B illustrates the table with measured values accompanying the graph in FIG. 4A.

FIG. 5A illustrates the results of a bridging ELISA (coating antibody AbCam UK, Catalogue No Ab-185924; HRP labelled detection antibody AbCam UK, Catalogue No Ab-635), for the detection of firefly luciferase in cellular extracts from an assay for the quantification of the ADCC activity of trastuzumab using iLite® effector cells expressing firefly luciferase under the control of a CD16a responsive promoter in the presence of iLite® target cells expressing Her2 compared with the detection of firefly luciferase activity using the One-Glo substrate (Promega). FIG. 5B illustrates the table with measured values accompanying the graph in FIG. 5A.

FIG. 6 Illustrates the Gyros ADA detection system on the left-hand side (LHS) of the Figure and the iLite® NAb assay on the upper right-hand side (RHS) of the Figure and on the lower RHS of the Figure the luciferase detection system based on the use of a pair of specific anti-luciferase antibody consisting of one molecule labelled with biotin bound to a streptavidin coated bead and a second anti-luciferase antibody molecule labelled with Alexa-647 that permits quantification of the biological activity detected by the cell-based iLite® NAb assay on the Gyros platform. Alternatively, unlabeled anti-luciferase anchor and detection antibodies may be used in conjunction with biotin or Alexa-647 labelled secondary antibodies.

FIG. 7 illustrates the profile of binding of the Alexa-647 labelled anti-luciferase detection antibody (AbCam UK, Catalogue No Ab-181640) to recombinant firefly luciferase in the Gyros detection system (FIG. 7A) and a dose response curve for the detection of recombinant firefly luciferase using the Gyros platform using high capacity porous beads and a standard spin speed (FIG. 7B).

FIG. 8 illustrates the results of an experiment to determine the optimal conditions for the detection of recombinant firefly luciferase on the Gyros immune detection system. FIG. 8A illustrates the profile of binding of the Alexa labelled anti-luciferase detection antibody (AbCam UK, Catalogue No Ab-635) to recombinant firefly luciferase in the Gyros detection system using either 3.0 or 30 μg of the capture antibody (AbCam UK, Catalogue No Ab-222862) and the photomultipliers set at 1%. FIG. 8B shows a dose response curve for the detection of recombinant firefly luciferase using the Gyros platform with high capacity porous beads using either 3.0, 10, or 30 μg of the capture antibody (AbCam UK, Catalogue No Ab-222862) using standard beads in a two-step protocol and the photomultipliers set at 1%.

FIG. 9 illustrates the detection of firefly luciferase in extracts of FGF-21 responsive cells treated with increasing concentrations of bFGF using the Gyros immune detection platform and an Alexa labelled anti-luciferase detection antibody (AbCam UK, Catalogue No Ab-181640) and 30 μg of the capture antibody (AbCam UK, Catalogue No Ab-222862) using high capacity porous beads in a two-step protocol and the photomultipliers set at 25%. Results are expressed as arbitrary units in FIG. 9A and as fold-induction relative to the control sample of FGF-21 responsive cells without bFGF treatment in FIG. 9B.

FIG. 10 illustrates the detection of firefly luciferase activity in extracts of FGF-21 responsive cells treated with increasing concentrations of bFGF using the One-Glo luciferase detection reagent (Promega) in FIG. 10A and firefly luciferase detected using the Gyros immune detection platform and an Alexa labelled anti-luciferase detection antibody (AbCam UK, Catalogue No Ab-181640) and 30 μg of the capture antibody (AbCam UK, Catalogue No Ab-222862) using high capacity porous beads in a two-step protocol and the photomultipliers set at 25% (FIG. 10B). Results are expressed as fold-induction relative to the control sample of FGF-21 responsive cells without bFGF treatment.

FIG. 11 Illustrates the MSD ADA detection system on the left-hand side (LHS) of the Figure and the iLite® NAb assay on the upper right-hand side (RHS) of the Figure and on the lower RHS of the Figure the luciferase detection system based on the use of a specific anti-luciferase antibody pair consisting of one molecule labelled with biotin and bound to a streptavidin coated plate and a second anti-luciferase antibody molecule labelled on its Fc moiety with a Sulfo-Tag that permits detection of the cell-based iLite™ NAb assay on the MSD platform.

FIG. 12 illustrates the results of an experiment designed to compare the efficacy of a monoclonal anti-firefly luciferase antibody MAI 16880 (Thermo Fischer) and a goat polyclonal anti-luciferase antibody Ab-181640 (AbCam, UK) for the detection of recombinant firefly luciferase in the MSD immune assay platform using the same anti-luciferase capture antibody AB-222862 (AbCam UK,) and either a Sulfo-Tag labelled goat anti-mouse (MSD, Catalogue No R32AC-5) or a donkey anti-goat (MSD, Catalogue No R32AG-5) secondary antibody respectively.

FIG. 13 illustrates the results of an experiment to compare the efficacy of different concentrations (0.1 or 0.3 μg/ml) of an anti-luciferase monoclonal antibody (AbCam UK, Catalogue No Ab-222862) as the capture antibody for the detection of recombinant firefly luciferase in the MSD immune assay platform used together with a polyclonal goat anti-firefly luciferase detection antibody (AbCam UK, Catalogue No Ab-181640) and a Sulfo-Tag labelled donkey anti-goat secondary antibody (MSD, Catalogue No R32AG-5).

FIG. 14 illustrates the results of an experiment to compare the efficacy of detection of a two-step procedure in which FGF-21 responsive cells were treated with increasing concentrations of bFGF in a micro-titer assay plate and incubated for 18 hours at 37° C. prior to lysis of the cells and transfer of the cell supernatants to the MDS plate, compared with the assay carried out directly on the MSD plate. An anti-luciferase capture antibody (AbCam UK, Catalogue No Ab222862) was used at a concentration of 0.1 μg/ml together with a goat anti-luciferase detection antibody (AbCam UK, Catalogue No Ab-181640) and a Sulfo-Tag labelled donkey anti-goat secondary antibody (MSD, Catalogue No R32AG-5).

FIG. 15 illustrates the results of an experiment comparing the efficacy of detection of recombinant firefly luciferase using the MSD detection platform in FIG. 15A with the detection of recombinant firefly luciferase activity using the Gyros immune detection platform in FIG. 15B. Results are expressed in arbitrary units.

FIG. 16 illustrates the results of an experiment comparing the efficacy of detection of recombinant firefly luciferase using the MSD detection platform in FIG. 16A with the detection of recombinant firefly luciferase activity using the Gyros immune detection platform in FIG. 16B. Results are expressed as fold induction relative to the control sample without recombinant firefly luciferase.

FIG. 17A illustrates the results of an experiment to determine the specificity of detection of recombinant firefly luciferase using a biotinylated monoclonal antibody directed against the V5 protein of Simian virus 5 bound to a streptavidin coated Biacore 3000 sensor chip.

FIG. 17B illustrates the results of an experiment for the detection of recombinant firefly luciferase using a biotin labelled monoclonal anti-luciferase antibody (Ab-222862, AbCam, UK) bound to a streptavidin coated Biacore 3000 sensor chip.

FIG. 18 illustrates the results of an experiment for the detection of recombinant firefly luciferase using a biotin labelled monoclonal anti-luciferase antibody (Ab-222862, AbCam, UK) bound to a streptavidin coated Biacore 3000 sensor chip. Results are expressed as respective difference in relative units relative to the control.

FIG. 19 illustrates a dose response curve for recombinant firefly luciferase using a biotin labelled monoclonal anti-luciferase antibody (Ab-222862, AbCam, UK) bound to a streptavidin coated Biacore 3000 sensor chip.

FIG. 20 illustrates a dose response curve and data analysis for recombinant firefly luciferase using a biotin labelled monoclonal anti-luciferase antibody (Ab-222862, AbCam, UK) bound to a streptavidin coated Biacore 3000 sensor chip.

FIG. 21 illustrates the PerkinElmer AlphaLISA ADA detection system on the left-hand side (LHS) of the Figure and the iLite™ NAb assay on the upper right-hand side (RHS) of the Figure and on the lower RHS of the Figure the luciferase detection system based on the use of a specific anti-luciferase antibody pair consisting of one molecule labelled on the Fc moiety with biotin and bound to a streptavidin coated donor bead and a second anti-luciferase antibody molecule bound via its Fc moiety to an acceptor bead labelled with digoxigenin that permits detection of the cell-based iLite™ NAb assay on the PerkinElmer AlphaLISA platform.

### SUMMARY OF THE INVENTION

Traditional cell-based potency assays for therapeutic agents or assays for the quantification of neutralizing antibodies directed against the drug or therapeutic agent are based on the quantification of an activity (or the inhibition of the activity) of a particular drug such as an effect on cell proliferation (simulation or inhibition), production of a cytokine or other soluble factor the activity of which can be readily measured, or induction of apoptosis, cytotoxicity etc. An alternative approach is to quantify signal transduction, that is directly related to the mechanism of action of a drug, using a reporter-gene following binding of a drug to a soluble target or cell surface target (receptor or other cell-surface bound molecule). The reporter-gene is activated by the same signal transduction pathway that is responsible for mediating the therapeutic action of the drug. Reporter-genes usually encode readily visible proteins that can be quantified using fluorescence or luminescence such as luciferases (firefly, Renilla, Gaussia, Nano Luciferase etc), fluorescent proteins such as green fluorescent protein (GFP) or dsRED or an enzyme such as chloramphenicol acetyltransferase (CAT) or a protease. The principal of the invention is that reporter-gene product or by-product produced either during the course of the cell-based assay or on conclusion of the assay is quantified either in the cell medium or in the supernatant of the cells for a secreted protein or following lysis of the cells with a passive lysis buffer containing a detergent such as NP40 or SDS such as those commercialized by Promega (Catalogue No E1941), New England Biolabs (catalogue No B3321S), or Biotium (catalogue No 99912). The reporter-gene product, such as firefly luciferase (FL) is then detected in the cell supernatant or cell lysate using a pair of antibodies (monoclonal or polyclonal) specific for the gene product such as a firefly luciferase pair labelled with the dual detection system specific for each assay platform as shown in the examples. Suitable anti-luciferase antibodies may in principle be any type of anti-luciferase antibody and may e.g. include: Anti-Luc (Photinus pyralis) mouse monoclonal antibody (AbD Serotec (Biorad) catalogue No MCA2076), anti-Luc (Photinus pyralis) mouse monoclonal (Genway Biotech catalogue No GWB-A18D67), Rabbit anti-Luc (Photinus pyralis) monoclonal antibody EPR17790 (AbCam catalogue No ab185924) and Alexa 488 labelled rabbit anti-Luc monoclonal antibody (AbCam catalogue No ab214950), anti-Luc (Photinus pyralis) mouse monoclonal antibody (Novus Biologicals, catalogue No NB 600-307), rabbit polyclonal anti-Renilla (Renilla reniformis) luciferase (Novus Biologicals, catalogue No NBP2-42914), rabbit monoclonal Anti-Renilla (Renilla reniformis) antibody (AbCam catalogue No 185925), Alexa Fluor 488 labelled rabbit anti-Renilla (Renilla reniformis) monoclonal antibody (AbCam catalogue No ab216113), biotin labelled rabbit polyclonal anti-Renilla (Renilla reniformis) antibody (Biorbyt, catalogue No orb196945), and anti-Gaussia princeps luciferase (ThermoFisher, catalogue No PA1181. The expression of the reporter-gene product such as e.g. firefly luciferase can be normalized with respect to the expression of a second reporter gene product such as e.g. Renilla luciferase or Nano Luciferase under the control of a constitutive promoter.

Thus, the invention permits for the first time biological activity detected using a cell-based assay to be quantified by ELISA or using an immune detection platform such MSD, Luminex, SMC, Alpco, AlphaLISA or Gyros, or a label-free detection systems such as SPR systems including Biacore.

Moreover, the invention permits for the first time complex cell based assays such as potency assays, the quantification of virus infectivity, or the quantification of the potency of a virus vector or virus transgene such as AAV transgene used in gene therapy, or assays for the detection of anti-drug neutralizing antibodies or neutralizing antibodies directed against a virus vector or transgene carried by the virus vector, or effector cell function such as ADCC, ADCP, or CDC to be analyzed by ELISA or on a high throughput immune-detection platform without the need to modify either the reporter-gene cell line and/or the assay platform. Furthermore, the same antibody, for example an anti-firefly luciferase monoclonal antibody, labelled with the dual detection system, may be used for all the different therapeutic agents used on a particular platform whereas currently a different antibody pair is required for each drug analyzed using a particular assay platform.

In a further embodiment of the invention in order to facilitate the detection and quantification of the ADCC activity of therapeutic antibodies the ADCC effector reporter-gene cell line and drug-specific target cells are mixed together with suitable dilution(s) of the drug to be analyzed. The effector cells (E) and target cells (T) may be either cultivated continuously in the laboratory or frozen separately and thawed immediately prior to use and then mixed at a suitable target cell ratio (E:T ratio) together with a suitable dilution(s) of the drug to be analyzed and incubated for an appropriate time prior to lysing the cells and addition of the FL specific antibody (monoclonal or polyclonal) pair and addition of a sample of the cell lysate supernatant to the assay platform for quantification of ADCC activity. Alternatively, the ADCC effector reporter-gene cell line and drug-specific target cells can be pre-mixed at the optimal effector (E): target cell (T) ratio prior to freezing the cells (“Combo”) either alone or together with a pair of anti-luciferase antibodies at the appropriate concentrations. Thus, upon thawing of the “Combo” vial of ADCC effector and drug-specific target cells frozen at the optimal E:T ratio are mixed directly with an appropriate dilution(s) of the drug for which ADCC activity is to be quantified and incubated for an appropriate time prior to lysing the cells and addition of the FL specific antibody (monoclonal or polyclonal) pair (in the case that the antibodies were not included in the vial of cells prior to freezing) and addition of a sample of the cell lysate supernatant to the assay platform for quantification of ADCC activity. The ADCP and/or CDC activity of therapeutic antibodies may be quantified in a similar manner using a target cell containing a protease cleavable reporter-gene product such as a luciferase that responds to the release of a protease and that once secreted can be quantified allowing the assessment of ADCP or CDC activity. Alternatively, the protease may induce a conformational change in the reporter-gene protein product that results in the appearance of a hidden epitope that can be detected by the antibody pair used in the platform detection system. This invention is also applicable to the quantification of the activity of cells used in adoptive therapy such as CAR-T cells using an automated assay platform. CAR-T activity would be quantified using the same type of target cell as that described above for an ADCP or CDC assay.

Suitable cell lines for use according to present invention may be found in e.g. WO 2004/039990, WO 2008/055153, PCT/EP2017/075053, PCT/EP2017/075055, and EP 18162485.9 which are hereby incorporated in their entirety by reference.

### Definitions

The terms “invalidated” or “muted” used interchangeably herein is meant to knock out a particular gene to ultimately change the phenotype of a cell. Effectively, the term is meant to encompass rendering a gene non-functional. An example may be the invalidation of a certain gene to remove the expression of a surface cell receptor.

The term “++” in relation to a “++ cell” is intended to mean a target cell in which the antigen/receptor is overexpressed. The terminology is used interchangeably herein with “T+”. Moreover, when the expression is used together with a receptor or antigen such as e.g. CD20++ is intended to mean that CD20 is overexpressed on the cell in question. As an example without intending to limit the scope of the invention, in the case of CD20 expression levels are increased some 16-fold on CD20++ target cells relative to the wild type CD20+ Raji cells

The term “−/−” in relation to “−/− cell” is intended to mean a target cell in which the antigen/receptor is not expressed, i.e. wherein the relevant gene has been knocked-out (invalidated) to mute the expression of the antigen/receptor in question. The terminology is used interchangeably herein with “T−”. Consequently, the cells no longer express detectable levels of the specific antigen recognized by the antibody since the gene encoding the specific antigen has been rendered nonfunctional. In the context of present invention this may be seen as a control target cell.

The term “E” is intended to mean “effector cells” and particularly effector cells according to the invention. The term “effector cell” is intended to mean any cell of any type that actively responds to a stimulus and effects some change (brings it about). One such example is cytokine-induced killer cells, strongly productive cytotoxic effector cells that are capable of lysing tumor cells. In a further example and in the context of present invention an effector cell is intended to mean any cell having Fc gamma receptors (FcγR or FCGR) on the surface of said cell which bind the Fc region of an antibody, wherein the antibody itself is specifically capable of binding to a target cell.

The term “T” is intended to mean a “target cell”, i.e. any cell having a specific receptor/antigen that reacts with a specific hormone, antigen, antibody, antibiotic, sensitized T cell, or other substance. In relation thereto the term “(T+)” is intended to mean antigen positive target cells and consequently a cell expressing an antigen on its surface and allowing for binding of an antibody. In contrast, the term “(T−)” is intended to mean an antigen negative target cells (control target cell) and consequently a cell not expressing an antigen on its surface and thus not capable of reacting with an antibody. Put differently, antigen −/− cells (or T− cells) do not express detectable levels of the specific antigen recognized by the antibody that is being tested for ADCC activity since the gene encoding the specific antigen has been rendered nonfunctional. Specifically, the target cells used according to the invention are the same type of cells which is in contrast to known methods which usually employ one cell type as the T+ cell and employs another cells type as the T− cell. Put differently, a homologous control target cells that is exactly the same cell identical in all respects as the antigen positive target (T+) cell except that it does not express the specific antigen recognized by the antibody being assayed. As mentioned above, this is in contrast to the use of a T-cell (T lymphocyte), for example, that is often used as a control target cell for the quanification of ritiximab activity using a CD20 expressing B-cell target cells.

The term “NHS” is intended to mean Normal Human Serum in e.g. a biological sample.

The term “automated assay” is meant to mean any assay platform that is designed to be used in an automated or at least partly automated setting such as e.g. the use of robotic processing and/or execution of the assay. The platform may in principle be any format, such as the use of multi-well systems such as e.g. microtiter plates or other formats comprising any type of containers. Typically, automated assays may be used as complete so-called handsfree (i.e. not requiring manual processing of the assay procedure) or walkaway assays which do not require manual monitoring of at least part of the assay procedure. The automation may comprise measuring and dispensing of various volumes of liquids in the respective containers and/or measuring the output of the assay for further processing and analysis.

Moreover, the term “automated assay” may also comprise a system in which multiple samples are analyzed simultaneously or in parallel for the presence of an analyte using an antibody directed against the analyte, the antibody being either in solution or attached to a surface either directly, or via the interaction between biotin and streptavidin, or indirectly using a secondary antibody, and detection of the analyte using either a second antibody labelled directly or indirectly in such a manner that it can be detected using the automated platform, or detection of the analyte by quantification of the change in a physical attribute such as mass or diffraction.

The term “immunoassay” is meant to mean any biochemical test that measures the presence or concentration of a macromolecule or a small molecule in a solution through the use of an antibody or an antigen.

The term “vector” or vector construct” refers to a DNA molecule used as a vehicle to transfer recombinant genetic material into a host cell. The four major types of vectors are plasmids, bacteriophages and other viruses, cosmids, and artificial chromosomes. The vector itself is generally a DNA sequence that consists of an insert (a heterologous nucleic acid sequence, transgene) and a larger sequence that serves as the “backbone” of the vector. The purpose of a vector which transfers genetic information to the host is typically to isolate, multiply, or express the insert in the target cell. Vectors called expression vectors (expression constructs) are specifically adapted for the expression of the heterologous sequences in the target cell, and generally have a promoter sequence that drives expression of the heterologous sequences. The choice of vector employed in embodiments of the present invention depends on the specific application of the vector encoding the polypeptides or polynucleotide.

The term “operatively linked” or “operably linked” refers to the connection of elements being a part of a functional unit such as a gene or an open reading frame. Accordingly, by operatively linking a promoter to a nucleic acid sequence encoding a polypeptide (an open reading frame, ORF) the two elements becomes part of the functional unit—a gene. The linking of the expression control sequence (promoter) to the nucleic acid sequence enables the transcription of the nucleic acid sequence directed by the promoter. By operatively linking two heterologous nucleic acid sequences encoding a polypeptide the sequences becomes part of the functional unit—an open reading frame encoding a fusion protein comprising the amino acid sequences encoding by the heterologous nucleic acid sequences. By operatively linking two amino acids sequences, the sequences become part of the same functional unit—a polypeptide. Operatively linking two heterologous amino acid sequences generates a hybrid (fusion) polypeptide.

### DETAILED DESCRIPTION OF THE INVENTION

Present invention relates to one or more cell lines. The cells may in principle be any cells, such as mammalian or non-mammalian cells, eukaryotic cells or prokaryotic cells. In one aspect the cells are mammalian cells such as e.g. human cells. In a further aspect the cells may be avian cells. Other non-limiting examples are vertebrate cells, plant protoplasts, fungal and yeast cells, and bacterial cells. Further non-limiting examples are Jurkat, Molt4, Raji, SKBR3, NK92, KHYG-1, HEK293 cells DT-40, PIL-5, or MSB-1.

In one aspect, present invention relates to a polynucleotide comprising a cis-acting regulatory sequence operably linked to a downstream promotor, wherein one or more of NF-AT, AP1, NFkB, STAT1, STAT3 and STAT5 is capable of binding to said cis-acting regulatory sequence. In one aspect the invention relates to a polynucleotide, wherein NF-AT, AP1, NFkB, and STAT5 are all capable of binding to said cis-acting regulatory sequence.

The polynucleotide according to the invention may comprise a promotor that is operable linked to an open read frame sequence encoding a first reporter protein. In one aspect, the reporter is an enzyme such as e.g. a luciferase or a fluorescent protein.

The polynucleotide according to the invention comprises a nucleotide sequence having at least about 70% sequence identity, such as e.g. at least about 75% sequence identity, such as e.g. at least about 80% sequence identity, such as e.g. at least about 85% sequence identity, such as e.g. at least about 90% sequence identity, such as e.g. at least about 95% sequence identity, such as e.g. at least about 98% sequence identity, such as e.g. at least about 99% sequence identity to SEQ ID NO.: 1 or a DNA sequence identical to SEQ ID NO.: 1, wherein SEQ ID NO.: 1 is GGAAGCGAAA ATGAAATTGA CTGGGACTTT CCGGAGGAAA AACTGTTTCA TACAGAAGGC GTGGATGTCC ATATTAGGAT GAGTCAGTGA CGTCAGAGCC TGATTTCCCC GAAATGATGA GCTAG.

The invention also relates to a vector construct comprising the polynucleotide according to the invention. The vector may be a plasmid or viral vector.

Present invention also relates to a cell comprising the vector as mentioned above, wherein said vector is episomal or integrated in the genome of said cell. The cell according to the invention may further expresses a second reporter protein which different from the first reporter protein.

Present invention also relates to a kit, comprising:

i) an effector cell (E) according to the invention which comprises a vector further comprising the polynucleotide with SEQ ID NO.: 1, capable of binding to the Fc region of an antibody;

ii) a cell (T−) in which the endogenous target/antigen to which said antibody is specific is invalidated (mutated) such that the target/antigen is not expressed by the cell; and

iii) a target cell (T+) in which the expression of the target to which said antibody is specific is enhanced or overexpressed.

The kit according to the invention may be such that, the cell in ii) and the cell iii) are exactly the same cell identical in all respects except the cell in ii) does not express a specific antigen recognized by the antibody or drug being assayed. The kit according to the invention, may be such that the target/antigen is one or more of CD20, mTNFα, erbB2, EGFR. Moreover, the kit according to the invention may comprise two vials and wherein the cells in i) and iii) are present in one and the same vial at the optimal E:T ratio. The ratio between the effector cell in i) and the target cell in iii) (E:T ratio) is in range from about 24:1 to about 2:1, or about 6:1, or about 3:1, or about 1.5:1.

Present invention also relates to a method for quantifying the Antibody-Dependent Cell-mediated Cytotoxicity (ADCC) activity ex vivo in clinical samples from patients treated with therapeutic antibodies, the method comprising the steps of;

a) contacting a sample obtained from a patient undergoing treatment comprising administration of an antibody, with target cells iii) according to the invention,

b) subtracting the signal obtained in the presence of cells ii) according to the invention, in which the drug target has been invalidated, from the signal obtained in the presence of effector cells i) according to the invention and target cells iii) according to the invention,

c) determining the ADCC activity on the basis of the signal relationship as measured in a) and b), and wherein a positive result for a serum sample (i.e. detectable ADCC activity) is present when the signal relationship for Effector cells i) & Target ++ cells iii)/Effector cells i) & Target −/− cells ii) ≥1 and wherein a negative result for a serum sample (i.e. no detectable ADCC activity) is present when the value for Effector cells i) & Target ++ cells iii)/value for Effector cells i) & Target −/− cells ii) ≤1, using the formula the formula [(E+T++Drug+NHS)−(E+T−+NHS)+(E+T+)]/E+T+, or [(E+T++NHS)−(E+T−+NHS)+(E+T+)]/E+T+, wherein (E) is the effector cells of any one of items 8-10, wherein the (T+) cells are the cells according to item 11 iii), wherein (T−) are the cells according to paragraph ii), and NHS=normal human serum sample.

In a further aspect, the invention also relates to a method for compensating for the non-specific increase in the reporter-gene signal in the presence of human serum, the method comprising: subtracting the signal obtained in the presence of cells i), i.e. cells according to the invention, from the signal obtained in the presence of effector cells i) and target cells iii) according to the invention that express the drug target or serum samples that exhibit activity that is not related to ADCC activity specific to the antibody under investigation when Effector cells i) & Target negative cells ii) >1, using the formula the formula [(E+T++Drug+NHS)−(E+T−+NHS)+(E+T+)]/E+T+, or [(E+T++NHS)−(E+T−+NHS)+(E+T+)]/E+T+, wherein (E) is the effector cells of the invention, wherein the (T+) cells are the cells according to the invention, wherein (T−) are the cells according to paragraph ii), and NHS=normal human serum sample.

In another aspect, the invention relates to an effector cell that has been engineered to over-express either the low affinity Fc receptor, FcγRIIIa (CD16A) V or F variants, or FcγRIIa (CD32) H or R variants that respond to ligation of the Fc moiety of antibody bound to the specific antigen expressed on target cells by activation of a NFAT responsive reporter gene, or by activation of a synthetic chimeric promoter containing binding sites for NF-AT, AP1, NFkB, and STAT5 operationally linked to the firefly luciferase (FL) reporter-gene as disclosed above.

In another aspect of the invention the effector cell has been engineered to over-express e.g. either the high affinity Fc receptor, FcγRI (CD64), or e.g. the low affinity Fc inhibitory receptor FcγRIIB1 (CD32), or the low affinity Fc inhibitory receptor FcγRIIB2 (CD32), or the low affinity Fc receptor Fc□RIIIB (CD16b) that respond to ligation of the Fc moiety of antibody bound to the specific antigen expressed on target cells by activation of a NFAT responsive reporter gene, or by activation of a novel synthetic chimeric promoter containing binding sites for NF-AT, AP1, NFkB, and STAT5 operationally linked to the firefly luciferase (FL) reporter-gene as disclosed herein.

In one aspect, the invention relates to a cell wherein the vector construct further comprises a polynucleotide encoding one or more selected from co-stimulatory molecule CD28, co-stimulatory molecule CD137 (4-1 BB), co-stimulatory molecule CD247 (T3 Zeta chain), co-stimulatory molecule CD278 (ICOS), or wherein co-stimulatory molecules are receptors selected from one or more of CD28, CD137L (4-1 BB), and ICOS.

In one aspect, the cell according to the invention is such that the one or more co-stimulatory molecules are expressed constitutively or over-expressed on the cells. The cells may further express CD16A or CD32.

In one aspect, the cells according to the invention are such that CTLA-4 (CD152) is specifically invalidated.

As mentioned above, the cells according to the invention may comprise a construct that expresses a first reporter protein. The first reporter protein may be an enzyme such as e.g. a luciferase or a fluorescent protein.

The cell may further be engineered to expresses a second reporter protein which different from the first reporter protein.

Moreover, the cells according to the invention may further express or overexpress an antigen recognized by an antibody or Fc fusion protein.

In a further aspect, the invention relates to a method for quantifying the Antibody-Dependent Cell-mediated Phagocytosis (ADCP) activity of therapeutic antibodies, the method comprising the steps of;

a) contacting a sample obtained containing an antibody, effector cells i) and with target cells iii) according to the invention,

b) subtracting the signal obtained in the presence of effector cells i) and cells ii) according to the invention, in which the drug target has been invalidated, from the signal obtained in the presence of effector cells i) according to the invention and target cells iii) according to the invention,

c) determining the ADCP activity on the basis of the signal relationship as measured in a) and b).

In one embodiment, the reporter gene (the first reporter gene) encodes an enzyme. In a preferred embodiment, the reporter (the first reporter) is a luciferase, such as firefly luciferase or Renilla luciferase.

In another embodiment, the reporter gene (the first reporter gene) encodes a fluorescent protein and the first reporter protein is a fluorescent protein. Useful fluorescent protein includes green fluorescent protein (GFP) and related fluorescent protein, e.g. enhanced green fluorescent protein (EGFP), yellow fluorescent protein (YFP), blue fluorescent protein (BFP) and variant there of displaying a different excitation/emission spectra.

Useful constitutively active promoters include but at not limited to cytomegalovirus (CMV) early enhancer/promoter, SV40 promoter, UBC promoter, PGK promoter, human β-actin (hACTB), human elongation factor-1α (hEF-1α), Thymidine Kinase (TK) promoter and cytomegalovirus early enhancer/chicken β-actin (CAG) promoters.

In a preferred embodiment, the first reporter protein is a luciferase and the second reporter (constitutively expressed) is also a luciferase with the proviso that the luciferases are not the same. In this way, the activity of both luciferases is readily detectable and can be read sequentially in the same well of an assay plate. For example, when the reporter gene construct produces firefly luciferase (first reporter protein), the constitutive production may be of a second luciferase (second reporter protein), e.g., Renilla luciferase. The activity of the first luciferase normalized relative to the activity of the second luciferase is described in US 2011/0189658 incorporated herein by reference. When conducting the assay, after the first reporter gene luciferase is measured, then a reagent is added to quench that specific luciferase so that any following reading will just read the luciferase from the constitutive construct, which then may be used for the purpose of normalization

In another aspect of the invention the cell, which may be a mammalian cell comprises (i) a first heterologous polynucleotide comprising a heterologous cis-acting regulatory sequence operably linked to a downstream promoter sequence, wherein said promoter is operably linked to an open reading frame encoding a first reporter protein,
(ii) a second heterologous polynucleotide encoding a chimeric transcription factor, wherein said chimeric transcription factor comprises the trans-activation domain of Elk-1 fused to a heterologous DNA binding domain capable of binding to said cis-acting regulatory sequence, and
(iii) a cell surface bound heterodimeric receptor protein comprising a tyrosine kinase FGFR1c and a beta-Klotho protein.

In one aspect, the DNA binding domain does not exist in mammalian cells.

In a further aspect, the heterologous DNA binding domain and its cognate cis-acting regulatory sequence may be of yeast or bacterial of origin.

The cis-acting regulatory sequence is an upstream activation sequence (UAS) and said heterologous DNA binding domain is a DNA binding domain capable of bind to said upstream activation sequence.

In one aspect, the cis-acting regulatory sequence is a galactose-responsive upstream activation sequence (UASG) and said heterologous DNA binding domain is the DNA binding domain galactose-responsive transcription factor GAL4 (GAL4DB).

In another aspect, the cis-acting regulatory sequence is the DNA binding site of LexA and said heterologous DNA binding domain is a DNA binding domain of repressor LexA protein.

The cell according to the invention may comprise a heterologous polynucleotide comprising promoter operably linked to a down-stream open reading frame encoding a beta-Klotho protein and from which said beta-Klotho protein is expressed, wherein said open reading frame encoding a beta-Klotho protein has been codon-optimized for expression in said cell.

In one aspect, the invention relates to a cell which may be a mammalian cell, wherein said open reading frame comprises or consist of the sequence set forth in SEQ ID NO.: 2 or a sequence having at least 75% sequence identity to SEQ ID NO.: 2, such as 79% sequence identity to SEQ ID NO.: 2, for example 85% sequence identity to SEQ ID NO.: 2, such as 90% sequence identity to SEQ ID NO.: 2, such as 95% sequence identity to SEQ ID NO.: 2, for example 97% sequence identity to SEQ ID NO.: 2, such as 98% sequence identity to SEQ ID NO.: 2, for example 99% sequence identity to SEQ ID NO.: 2.

The first reporter protein may be an enzyme, wherein said first reporter protein may be a luciferase.

As mentioned above, the cell according to the invention may further expresses a second reporter protein, wherein said second reporter protein is expressed from a constitutive promoter.

In one aspect, cell according to the invention may be a cell line selected from e.g. the group comprising HEK293, Jurkat, K652 and U937.

In a further aspect, the cell according to the invention may be a cell line selected from HEK293.

The invention also relates to a cell as disclosed in WO 2004/039990 and/or WO 2008/055153 which are hereby incorporated by reference in their entirety.

Consequently, present invention also relates to a cell transformed with a reporter gene construct comprising a nucleotide sequence encoding a reporter gene product operatively linked to one or more transcriptional control elements that is regulated by the signal transduction activity of a cell surface protein in response to an extracellular signal, said cell having been treated such that it will maintain said signal transduction activity for at least about 1 hour but no more than about 30 days at a temperature above freezing before losing said signal transduction activity.

The cell may be in a frozen state, wherein said treatment as mentioned above has occurred substantially immediately prior to the cell having been frozen.

The cells may have been treated with an anti-mitotic and pro-apoptotic agent and then re-suspended in a solution containing a cryopreservative such that said cells maintain said signal transduction activity for at least about 1 hour but no more than about 30 days at a temperature above freezing before losing said signal transduction activity.

The treated cells may have been frozen at about −80° C. substantially immediately after having been treated and then re-suspended in a solution containing a cryopreservative.

The cryopreservative may be e.g. dimethylsulfoxide (DMSO) or any other cryopreservative known in the art, such as e.g. glycerol.

In one aspect, the cryopreservative may be in an amount of about e.g. 10% DMSO in the solution.

Furthermore, the cryopreservative may be a combination of about 2.5% dimethylsulfoxide (DMSO) and 10% glycerol.

In one aspect, the anti-mitotic and pro-apoptotic agent may be vinblastine.

In another aspect the anti-mitotic and pro-apoptotic agent may be 5-fluorouracil.

The cells according to the invention may have been irradiated with γ radiation at an intensity and for a sufficient amount of time such that said cells maintain said signal transduction activity for at least about 1 hour but no more than about 30 days at a temperature above freezing before losing said signal transduction activity.

The intensity and amount of time of γ radiation may be about e.g. 6 to about e.g. 12 (Gy).

The temperature above freezing may be room temperature.

The surface protein may be a cell surface receptor, wherein said cell surface receptor is selected from a cytokine receptor, a growth factor receptor, a hormone receptor, a neuro receptor, a T cell receptor, an antigen receptor, and a complement receptor.

In one aspect, the cell surface receptor may be a Type I interferon receptor and said extracellular signal is provided by a Type I interferon.

In a further aspect, the one or more transcriptional control elements may comprise an interferon stimulatory response element (ISRE), wherein said ISRE comprises the nucleotide sequence of SEQ ID NO: 3.

On one hand, the cell surface receptor may be a Type II interferon receptor and said extracellular signal is provided by a Type II interferon.

In one aspect, the one or more transcriptional control elements may comprise a gamma activated sequence (GAS).

In one aspect of the invention, the cell surface receptor is an interferon receptor and wherein the extracellular signal may be provided by a Type I interferon and/or a Type II interferon.

In a further aspect, the one or more transcriptional control elements may comprise an interferon stimulatory response element (ISRE) and a gamma activated sequence (GAS).

According to the invention the reporter gene product may be selected from the group comprising e.g. firefly luciferase, bacterial luciferase, jellyfish aequorin, enhanced green fluorescent protein (EGFP), chloramphenicol acetyltransferase (CAT), dsRED, ss-galactosidase, and alkaline phosphatase.

In one aspect, the reporter gene product is firefly luciferase.

In another aspect, the reporter gene product is enhanced green fluorescent protein (EGFP).

In a further aspect, the reporter gene product is jellyfish aequorin.

In one aspect the cell may be a PIL5 cell.

In a further aspect, the invention also relates to a cell line transformed with a reporter gene construct comprising a nucleotide sequence encoding a reporter gene product operatively linked to a transcriptional control element that is activated as part of the signal transduction pathway initiated by a first cell surface molecule or complex in response to a first extracellular signal, which signal transduction pathway includes a transcription factor that binds to the transcriptional control element so as to activate said transcriptional control element and thereby regulate transcription of the reporter gene, the improvement whereby the sensitivity and/or the specificity of the response of the cell line to the extracellular signal is improved, wherein: a) said transcription control element is a modification of a naturally occurring transcriptional control element that is activated as part of the signal transduction pathway initiated by said first cell surface molecule or complex in response to said first extracellular signal, or is a synthetic promoter comprising an optimal number of response elements specific for said transcriptional factor activated by said first cell surface molecule or complex but lacking response elements for other transcription factors, such that the sensitivity and/or specificity of the transcriptional control element is improved relative to the naturally occurring transcriptional control element; and/or b) the cells of said cell line lack a second cell surface molecule that responds to, or is part of a complex that responds to, a second extracellular signal, which second extracellular signal, if said second cell surface molecule were present, would cause the initiation of a signal transduction pathway that modulates the transcription of said reporter gene.

The modification of a naturally occurring transcriptional control element may be obtained by site directed mutagenesis of said naturally occurring transcriptional control element and selection for a modification that improves the sensitivity and/or specificity of the naturally occurring transcriptional control element.

Moreover, the modification of a naturally occurring transcriptional control element may comprise a synthetic nucleotide sequence that comprises a tandem repeat of the naturally occurring or consensus sequence of the binding site for said transcription factor while lacking binding sites for other transcription factors.

In one aspect, the transcription factor is NFKβ.

In one aspect the tandem repeat of the binding site for transcription factor NFKB consists of the sequence of SEQ ID NO: 4.

In one aspect, the cell line naturally exists without said second cell surface molecule.

In a further aspect the first extracellular signal may be tumor necrosis factor α (TNFα) and said second extracellular signal may be interferon-γ (IFNγ) and/or interleukin-2 (IL2).

The cells of the cell lines according to the invention may have been genetically engineered to knock out the said second cell surface molecule.

In a further aspect, the first extracellular signal may be interferon-[gamma] (IFN[gamma]) and said second extracellular signal is interferon-[alpha] (IFN[alpha]) and/or interferon-[beta] (IFN[beta]).

In yet a further aspect, at least the extracellular portion of said first cell surface molecule or complex is that of a first species cell surface molecule or complex and the cells of said cell line are cells of a second species that have been genetically engineered to knock in said first cell surface molecule or complex.

In one aspect the first and/or second cell surface molecule or complex may be a cell surface receptor, wherein said first and/or second cell surface molecule or complex is a pattern recognition receptor.

Consequently, present invention also relates to use of a cell or cell line according to the invention in an automated assay or assay platform. It is to be understood that an automated assay may include any platform used for partially or wholly automated assays or procedures for performing assays in any form. Non-limiting examples are any assays such as ELISA in any context, automated assay platforms such as e.g. the Gyros, MSD, or AlphaLISA systems, or by label-free detection using SPR such as the Biacore system.

The advantage with present invention is that the cells disclosed herein need not be modified and/or the assay platforms need not be modified or adapted for use of the cell according to the invention, while still being able to provide reliable, robust and stable assays and results thereof.

### EXAMPLES

### Example 1

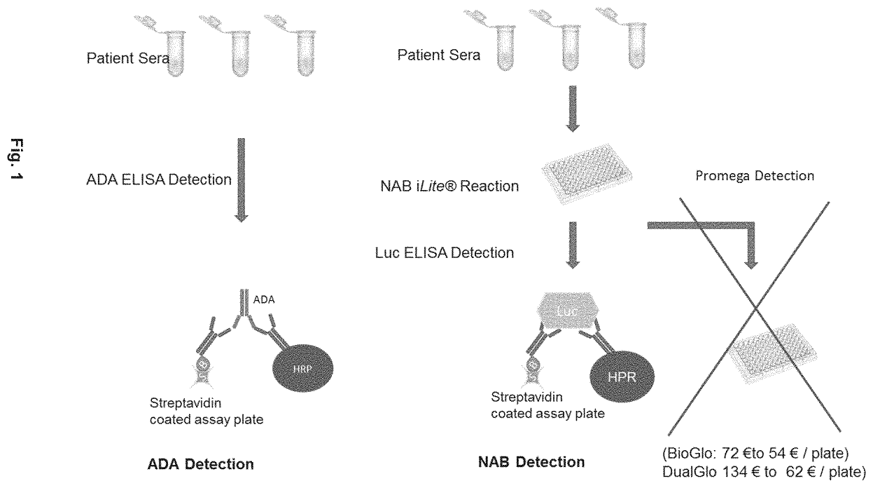

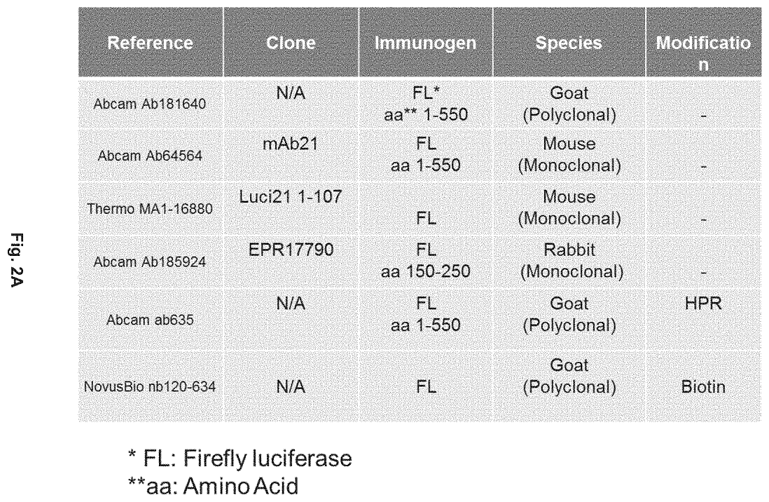

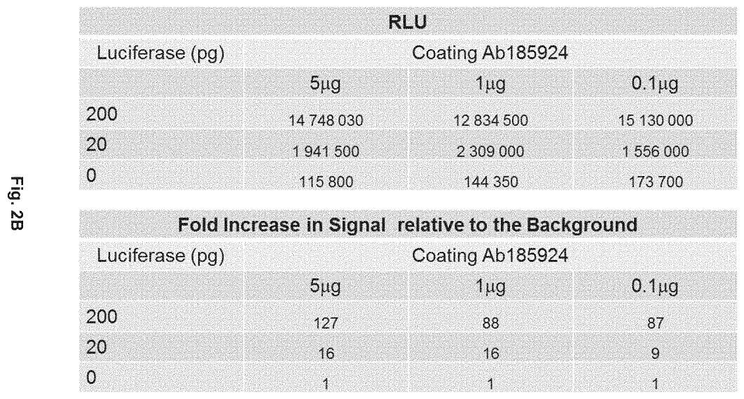

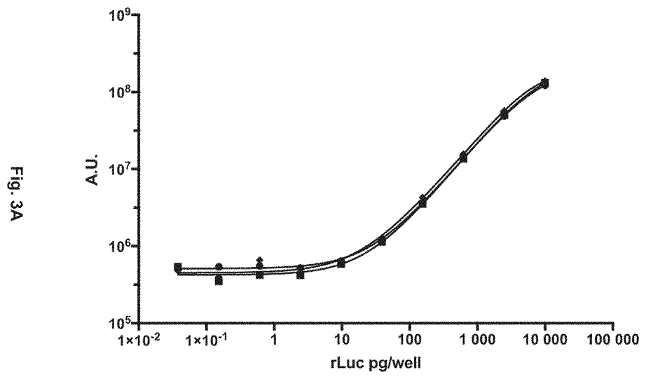

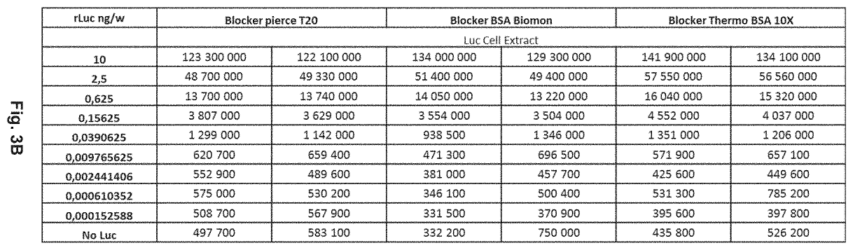

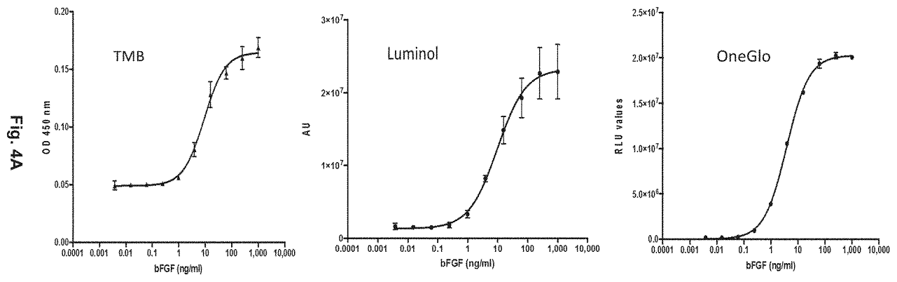

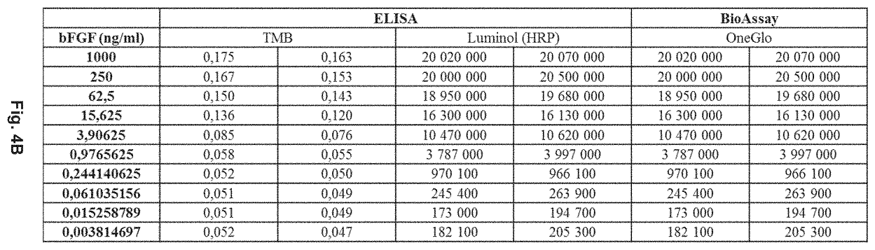

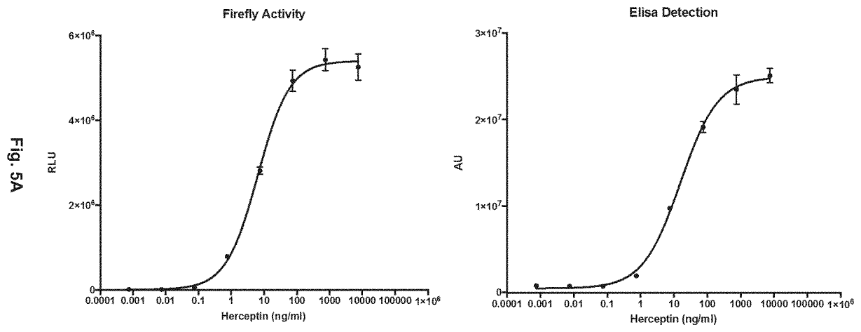

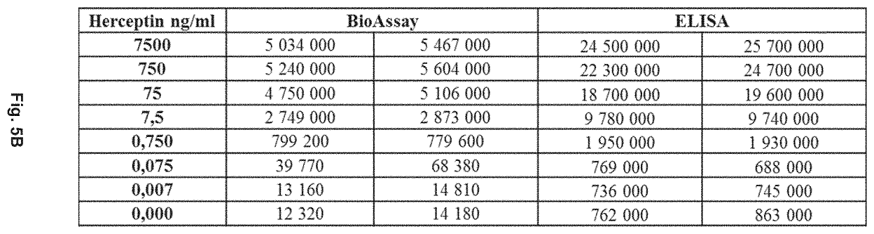

The bridging ELISA in which a drug (small molecule, peptide, or biopharmaceutical) is detected by the formation of a bridge between the drug, and one molecule of an antibody (monoclonal or polyclonal) directed against one epitope of the drug attached either directly to a solid surface, usually a 96, or 384-well micro-titer plate, or an antibody (monoclonal or polyclonal) directed against one epitope of the drug labelled with biotin that it is turn bound to a streptavidin coated surface, usually a 96, or 384-well micro-titer plate or streptavidin coated bead, and another molecule of an antibody (monoclonal or polyclonal) directed against a second epitope of the drug labelled with horse radish peroxidase (HRP) or another suitable marker is used widely for the quantification of the level a drug (FIG. 1). Similarly, the presence of anti-drug antibodies in a sample can be detected by the formation of a bridge between two molecules of the drug and one molecule of an antibody (monoclonal or polyclonal) directed against one epitope of the drug attached either directly to a solid surface, usually a 96, or 384-well micro-titer plate, or an antibody (monoclonal or polyclonal) directed against the drug labelled with biotin that it is turn bound to a streptavidin coated surface, usually a 96, or 384-well micro-titer plate or streptavidin coated bead, and another molecule of an antibody (monoclonal or polyclonal) directed against a second epitope of the drug labelled with horse radish peroxidase (HRP) or another suitable marker is used widely for the quantification of anti-drug antibodies (FIG. 1). Similarly, both the primary anchor and detection anti-luciferase antibodies may be used in conjunction with secondary detection antibodies (monoclonal or polyclonal) directed against the primary anchor and detection anti-luciferase antibodies. A series of 6 commercially available antibodies directed against firefly luciferase (FIG. 2A) were tested for their ability to bind recombinant firefly luciferase (FIG. 2B). Results are expressed as the fold increase in the signal relative to the background reading without luciferase, when used as either the capture or detection antibody compared with the values obtained for the quantification of firefly luciferase activity (FIG. 2B) using a commercially available substrate (One-Glo, Promega). The optimum combination for the detection of recombinant firefly luciferase was found to be antibody Ab-185924 (AbCam, UK) used as the coating antibody at a concentration of 100 ng/well and antibody Ab-635 labelled with HRP (AbCam, UK) used at a dilution of 1/20,000 as the detection antibody (FIG. 2B). No significant differences were observed between the use of an in-house blocking buffer and two commercially available blocking buffers (FIG. 3). HRP could be quantified using either a 3,3′,5,5′-tetramethylbenzidine (TMB) substrate and quantification of the signal using a luminometer or by chemiluminescence using a luminol substrate that allowed a level of detection to be obtained comparable to that obtained measuring luciferase activity with the One-Glo (Promega) substrate (FIG. 4). FGF-21 responsive cells containing the firefly luciferase reporter-gene under the control of a FGF-21 and bFGF responsive promoter were treated with increasing concentrations of bFGF in a micro-titer assay plate and incubated for 18 hours at 37° C. prior to quantification of firefly luciferase activity using One-Glo (Promega, catalogue No E6110) and lysis of the cells and quantification of firefly luciferase using a biotin labelled anti-luciferase capture antibody (AbCam UK, Catalogue No Ab-185924) and a HRP labelled anti-luciferase detection antibody (AbCam UK, Catalogue No Ab-635) as shown in FIG. 4. Results are expressed as fold-induction relative to the control sample of FGF-21 responsive cells without bFGF treatment in the left-hand panel and as arbitrary units in the right-hand panel (FIG. 5). The use of anti-firefly luciferase antibodies to detect firefly luciferase in extracts of FGF-21 responsive cells treated with increasing concentrations of bFGF allowed a level of detection to be obtained comparable to that obtained measuring luciferase activity with the One-Glo (Promega) substrate (FIG. 4).

The activity of numerous therapeutic antibodies is mediated in part by immune-mediated effector cell function such as antibody dependent cellular cytotoxicity (ADCC), or antibody dependent cellular phagocytosis (ADCP), following binding of the variable regions of the antibody to a specific antigen on the surface of target cells and the interaction of the Fc moiety of the antibody with a Fc receptor on an immune effector cell (1,2). Quantification of ADCC activity requires the use of cell-based assays to assess target cell cytotoxicity or effector cell reporter-gene activity following binding of the Fc moiety of the antibody to the FcγRIIIA receptor (CD16a), on the effector cells and binding of the antibody to a specific antigen on the target cells (1,2). Extracts of engineered Jurkat ADCC effector cells (3) expressing firefly luciferase under the control of a CD16 responsive promoter and HER2 positive target cells (3) at an effector: target cell (E:T) ratio of 3:1 were incubated with increasing concentrations of trastuzumab (Herceptin®) for 4 hours at 37° C. prior to quantification of firefly luciferase activity using One-Glo (Promega, catalogue No E6110) and lysis of the cells and quantification of firefly luciferase using an anti-luciferase capture antibody (AbCam UK, Catalogue No Ab-185924) and a HRP labelled anti-luciferase detection antibody (AbCam UK, Catalogue No Ab-635).

The use of anti-firefly luciferase antibodies to detect firefly luciferase in extracts of Jurkat ADCC effector cells expressing firefly luciferase under the control of a CD16 responsive promoter in the presence of HER2 positive target cells allowed the ADCC activity of trastuzumab to be determined with results comparable to those obtained measuring luciferase activity with the One-Glo (Promega) substrate (FIG. 5). These results show for the first time how complex biological processes such as immune-mediated effector cell function such as ADCC or ADCP can be quantified using a simple immune detection procedure.

### Example 2

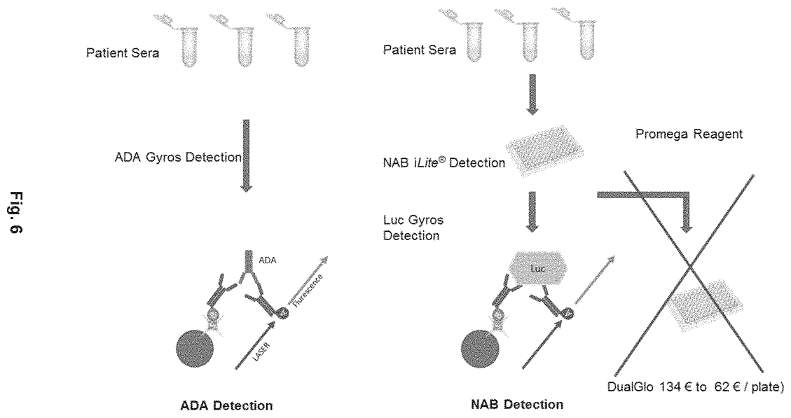

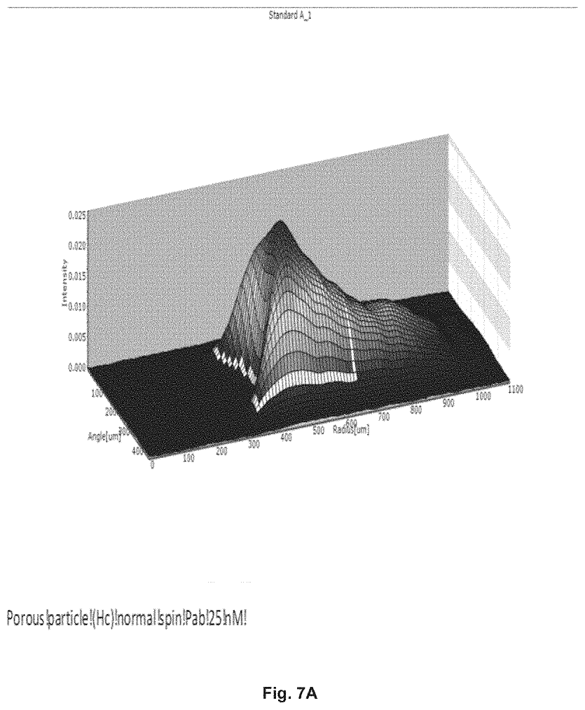

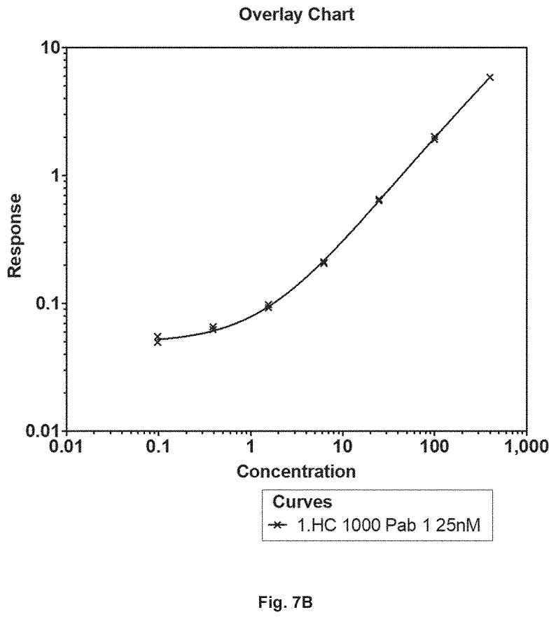

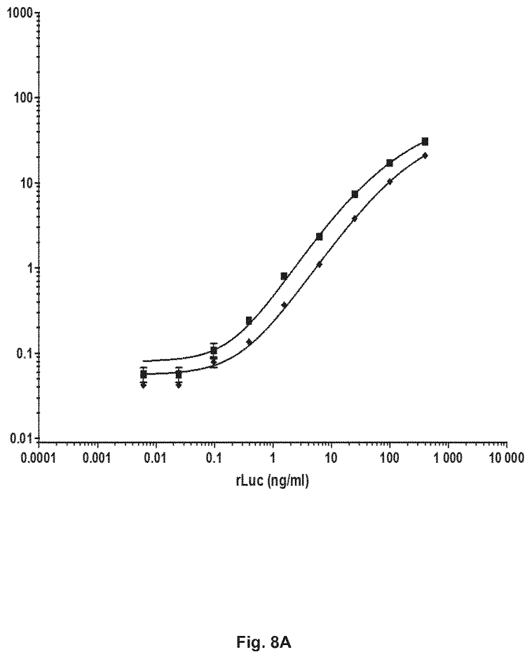

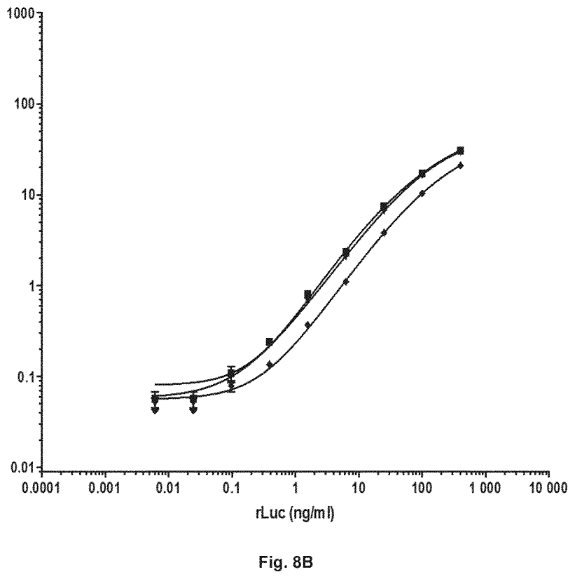

The Gyros platform technology is based on the use of centrifugal control of capillary action using a CD engineered to incorporate nanoliter microfluidics and a detection system based on laser activated fluorescence. Immunogenicity assays using the Gyros platform are based on a bridging ELISA in which anti-drug antibodies are detected by the formation of a bridge between two molecules of the drug labelled with biotin and another molecule of the drug labelled with a florescent marker such as Alexa-647 (FIG. 6). The presence of anti-drug antibodies in a sample will form a bridge allowing the Alexa labelled drug molecule to be bound to the biotin labelled drug molecule that it is turn bound to a streptavidin coated bead. The fluorescence emitted by the Alexa labelled bound drug is then quantified following activation with a laser. Alternatively, the potency of a therapeutic antibody may be quantified using an anti-drug antibody pair, specific for the therapeutic antibody, labelled with biotin and Alexa that detects the drug that forms a bridge between the two anti-drug antibodies molecules (monoclonal or polyclonal). Similarly, both the primary anchor and detection anti-luciferase antibodies may be used in conjunction with secondary anchor and detection antibodies (monoclonal or polyclonal). A different dual labelled drug pair is required for each Gyros ADA assay. In contrast, a single dual labelled anti-luciferase antibody pair can be used for the quantification of the potency, NAb response, and effector cell response for all the drugs tested using the invention that allows cell-based assays to be quantified using the Gyros platform. A biotin labelled anti-luciferase anchor antibody Ab-222862 (AbCam, UK) together with an Alexia 647 labelled an anti-luciferase detection antibody Ab-181640 (AbCam, UK) were used to detect increasing concentrations recombinant firefly luciferase using the Gyros platform. The profile of detection of fluorescence revealed that the Alexia-647 labelled an anti-luciferase detection antibody employed was of only moderate affinity (FIG. 7A). Notwithstanding the limited affinity of the detection antibody employed a dose response curve comparable to that obtained when monitoring firefly luciferase activity was obtained enabling biological activity to be quantified using the Gyros immune detection platform and a pair of anti-luciferase antibodies (FIG. 7B). Different concentrations of a biotin labelled anti-luciferase anchor antibody Ab-222862 (AbCam, UK) together with an Alexia-647 labelled an anti-luciferase detection antibody Ab-181640 (AbCam, UK) were used to determine the optimum conditions for the detection of recombinant firefly luciferase using the Gyros platform with the photomultipliers set at 1% (FIG. 8).

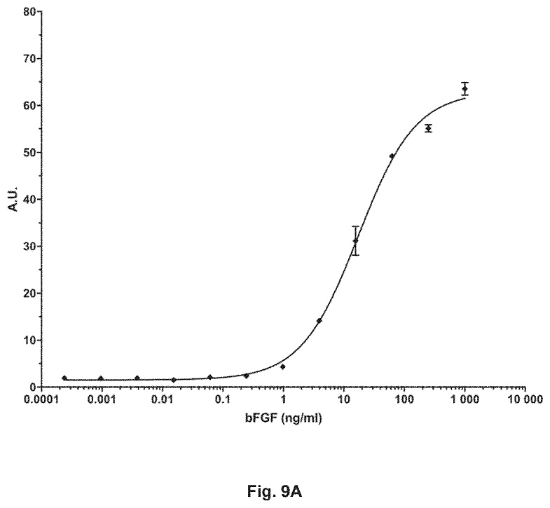

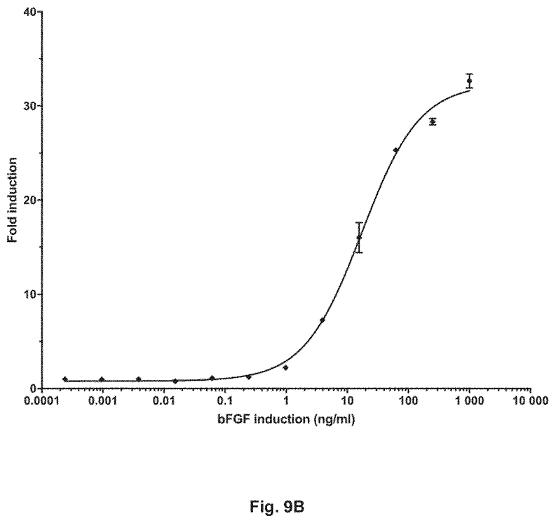

The Gyros immune detection platform was used to detect firefly luciferase in extracts of FGF-21 responsive cells containing the firefly luciferase reporter-gene under the control of a FGF-21 and bFGF responsive promoter following treatment of the cells with increasing concentrations of bFGF for 18 hours at 37° C. prior to quantification of firefly luciferase activity using One-Glo (Promega, catalogue No E6110) and lysis of the cells and quantification of firefly luciferase using an anti-luciferase capture antibody Ab-222862 (AbCam, UK) and an Alexia-647 labelled an anti-luciferase detection antibody Ab-181640 (AbCam, UK. Results are expressed as arbitrary units in FIG. 9A and as fold-induction relative to the control sample of FGF-21 responsive cells without bFGF treatment in FIG. 9B. The use of anti-firefly luciferase antibodies to detect firefly luciferase in extracts of FGF-21 responsive cells treated with increasing concentrations of bFGF allowed a level of detection to be obtained using a pair of anti-luciferase antibodies in the Gyros platform (FIG. 10A) comparable to that obtained measuring luciferase activity with the One-Glo (Promega) substrate (FIG. 10B).

### Example 3

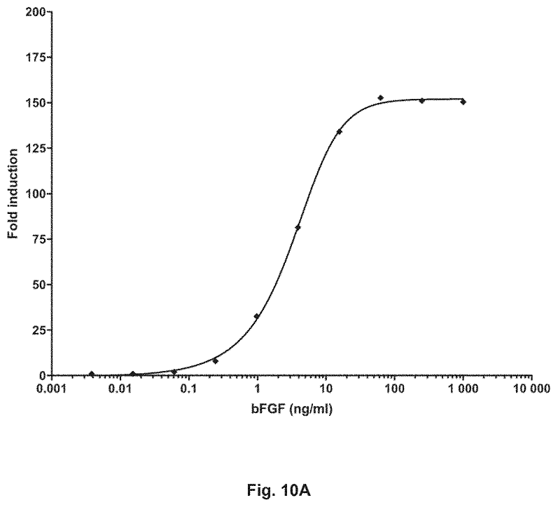

The MSD platform technology is based on the use of electrochemiluminescence and a detection system based on a Sulfo-Tag labelled antibody that detects an analyte bound to a capture antibody that is in turn bound to a carbon or gold coated 96-well plate with an embedded electrode. Immunogenicity assays using the MSD platform are based on a bridging ELISA in which anti-drug antibodies are detected by the formation of a bridge between two molecules of the drug consisting one molecule of which is labelled with biotin bound to a streptavidin coated gold plate and another molecule of the drug (a monoclonal or polyclonal therapeutic antibody) with a Sulfo-Tag (FIG. 11). The presence of anti-drug antibodies in a sample will form a bridge allowing the Sulfo-Tag labelled drug molecule to be bound to the biotin labelled drug molecule that it is turn bound to a streptavidin coated plate. The light signal emitted by the Sulfo-Tag labelled bound drug is then quantified. Alternatively, the potency of a therapeutic antibody may be quantified using an anti-drug antibody pair, specific for the therapeutic antibody, labelled with biotin and the Sulfo-Tag that detects the drug that forms a bridge between the two anti-drug antibodies molecules (monoclonal or polyclonal). Similarly, both the primary anchor and detection anti-luciferase antibodies may be used in conjunction with secondary anchor and detection antibodies (monoclonal or polyclonal). A different dual labelled drug pair or primary antibody pair is required for each MSD ADA assay. In contrast, a single dual labelled anti-luciferase antibody pair can be used for the quantification of the potency, NAb response, and effector cell response for all the drugs tested using the invention that allows cell-based assays to be quantified using the MSD platform.

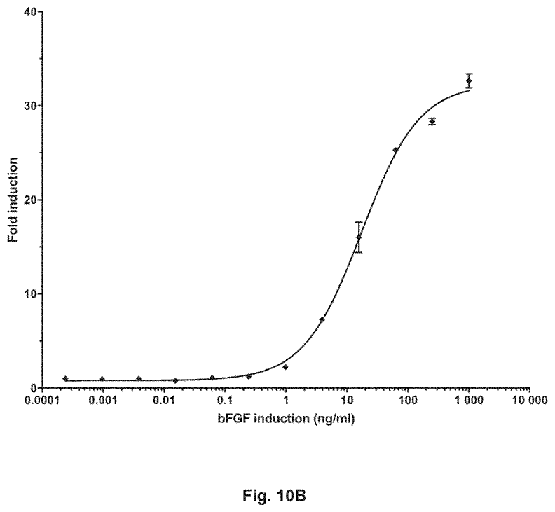

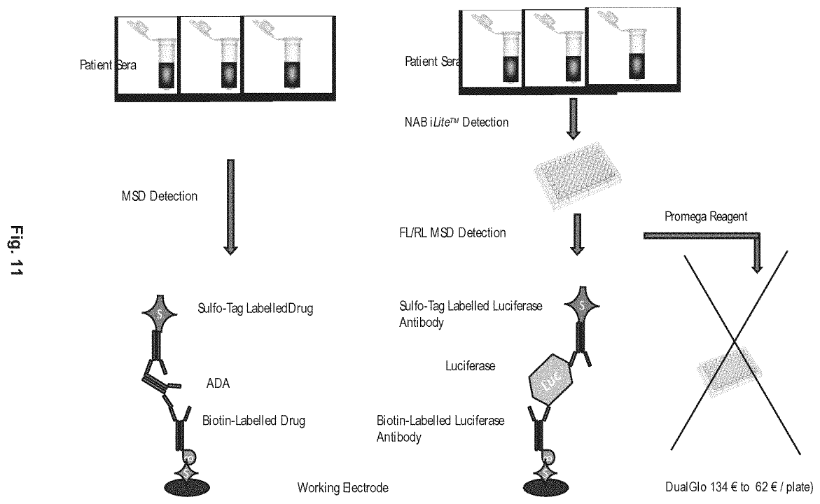

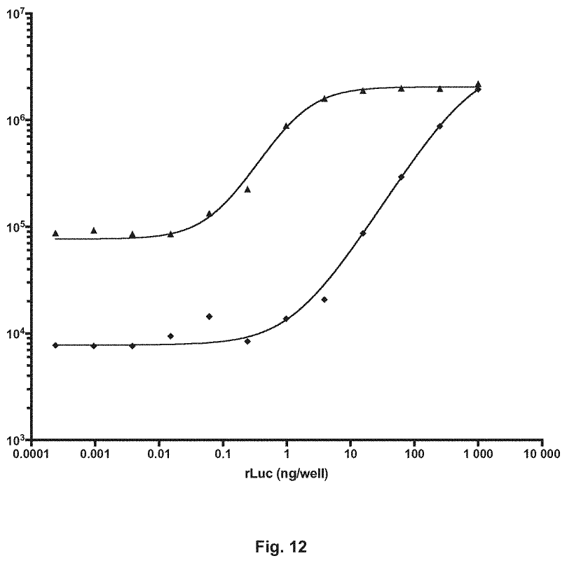

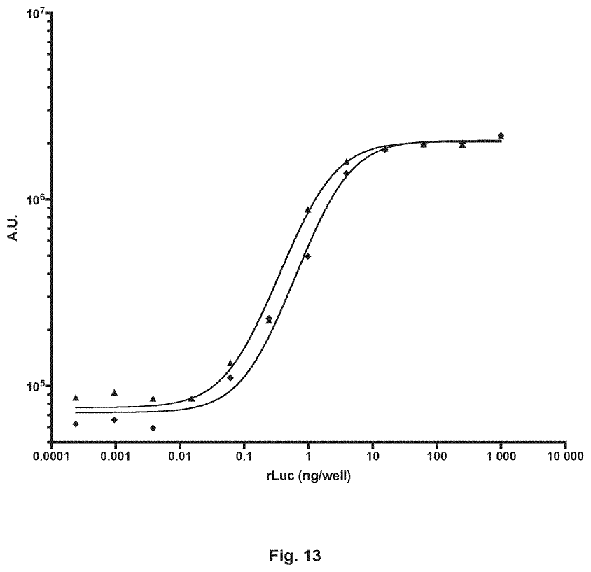

Two different anti-luciferase antibodies (monoclonal or polyclonal) were tested for their relative efficacy when used to capture recombinant luciferase in the MSD immune assay platform when used together with a goat anti-luciferase detection antibody (AbCam UK, Catalogue No Ab-635) and a Sulfo-Tag labelled donkey anti-goat secondary antibody (MSD, Catalogue No R32AG-5). The results show clearly that when both antibodies were used at a concentration of 0.3 μg/ml antibody Ab-181640 (AbCam, UK) gave superior results (FIG. 12). The use of antibody Ab-181640 at a concentration of either 0.1 or 0.3 μg/ml to capture recombinant firefly luciferase in the MSD immune assay platform used together with an anti-firefly luciferase detection antibody (AbCam UK, Catalogue No Ab-635) and a Sulfo-Tag labelled donkey anti-goat secondary antibody (MSD, Catalogue No R32AG-5) resulted in a dose-response curve (FIG. 13) similar to that obtained when for the quantification of firefly luciferase activity.

The efficacy of detection of a two-step procedure in which FGF-21 responsive cells were treated with increasing concentrations of bFGF and incubated for 18 hours at 37° C. in a micro-titer assay plate prior to lysis of the cells and transfer of the cell supernatants to the MDS plate was compared with that obtained when the assay was carried out directly on the MSD plate using two concentrations (0.1 or 0.3 μg/ml) of an anti-luciferase capture antibody (AbCam UK, Catalogue No Ab181640) and a Sulfo-Tag labelled anti-luciferase detection antibody (AbCam UK, Catalogue No Ab365). The results show clearly that the one-step procedure in which the FGF-21 responsive cells are treated with increasing concentrations of bFGF directly in the MSD detection plate is equivalent to a two-stem procedure in which cells are first treated in an assay plate and cell lysates are then transferred to the MSD assay plate (FIG. 14).

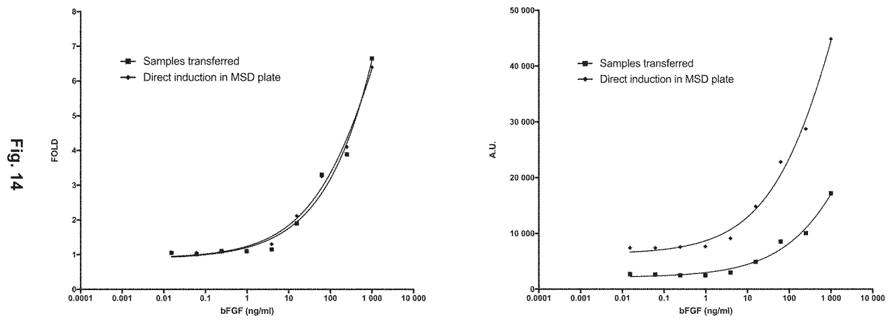

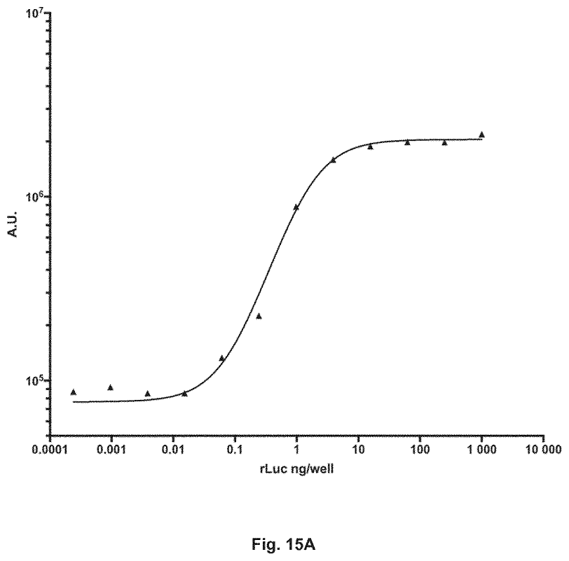

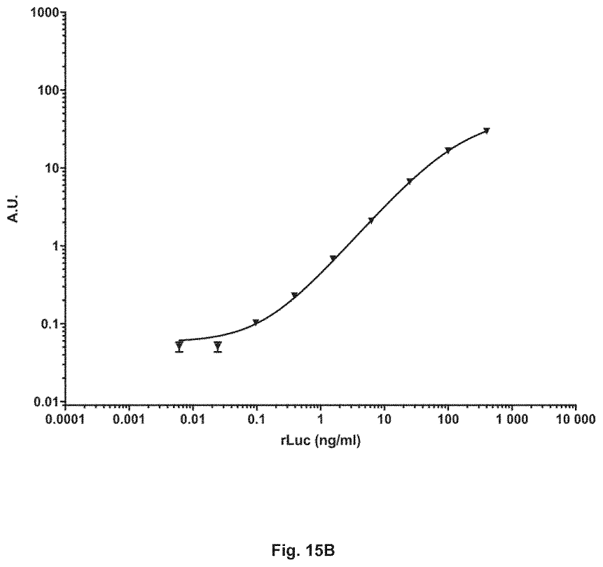

The efficacy of detection of recombinant firefly luciferase using the MSD detection platform was compared with that using the Gyros immune detection platform. The results obtained using the MSD system (FIG. 15A) were comparable to those obtained using the Gyros platform (FIG. 15B) whether results were expressed in arbitrary units (FIG. 15) or as fold induction relative to the control sample without firefly luciferase (FIG. 16).

### Example 4

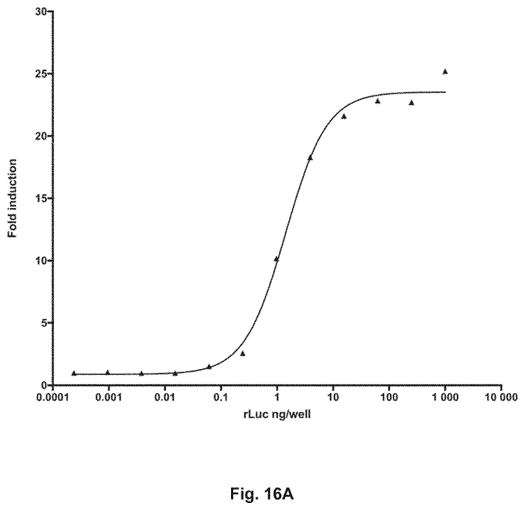

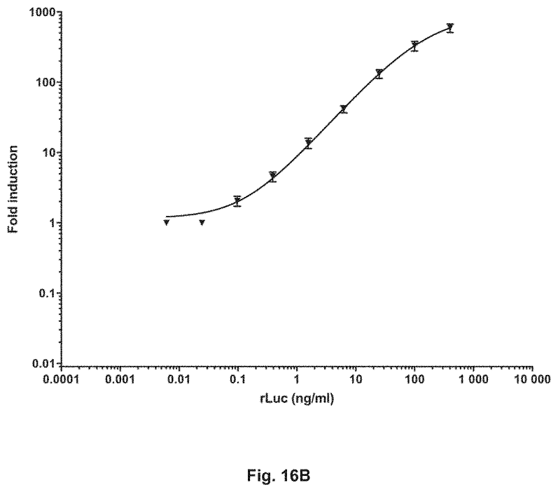

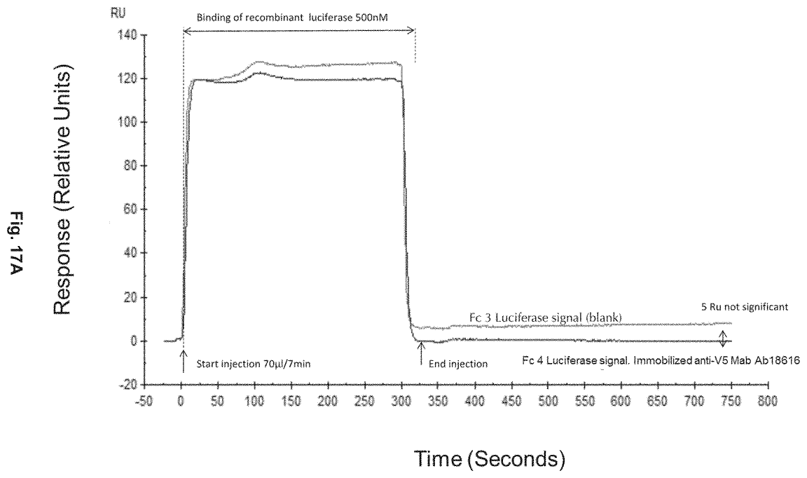

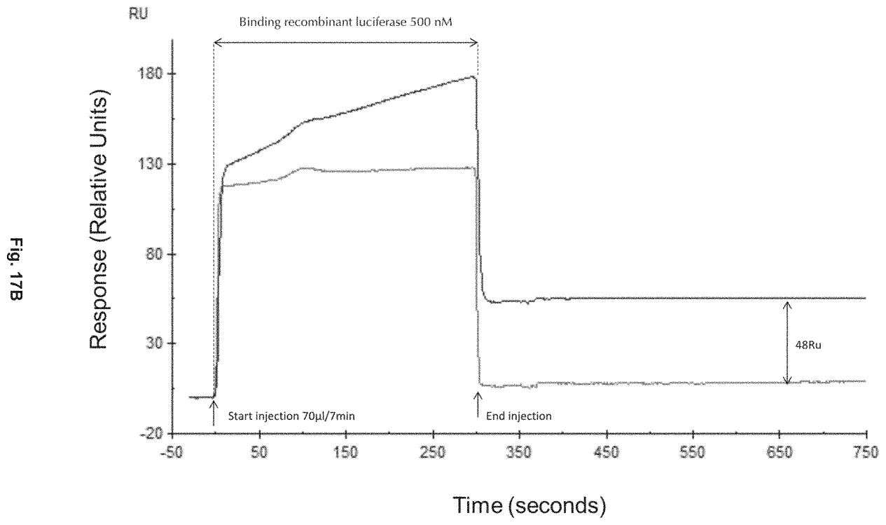

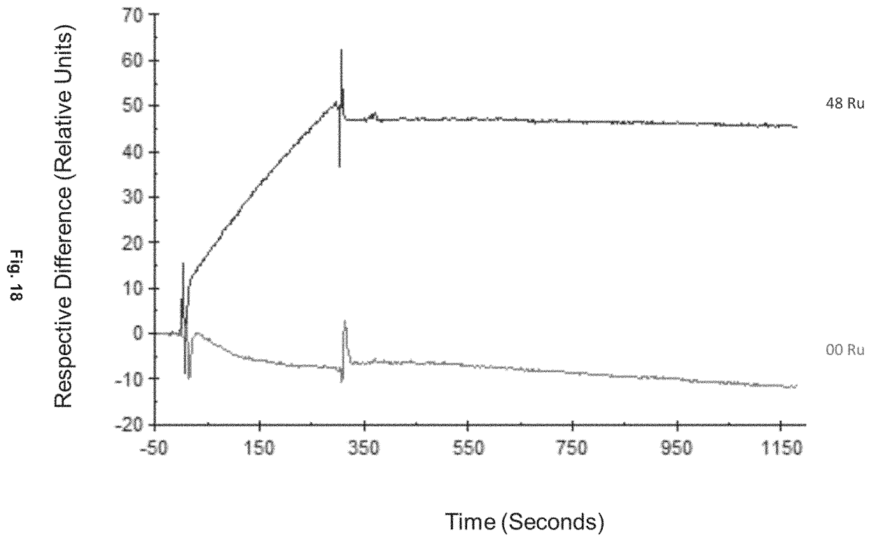

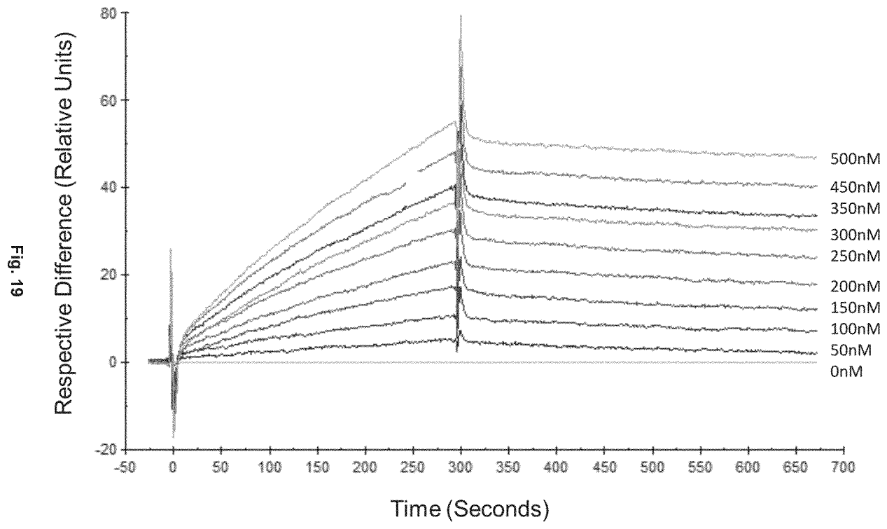

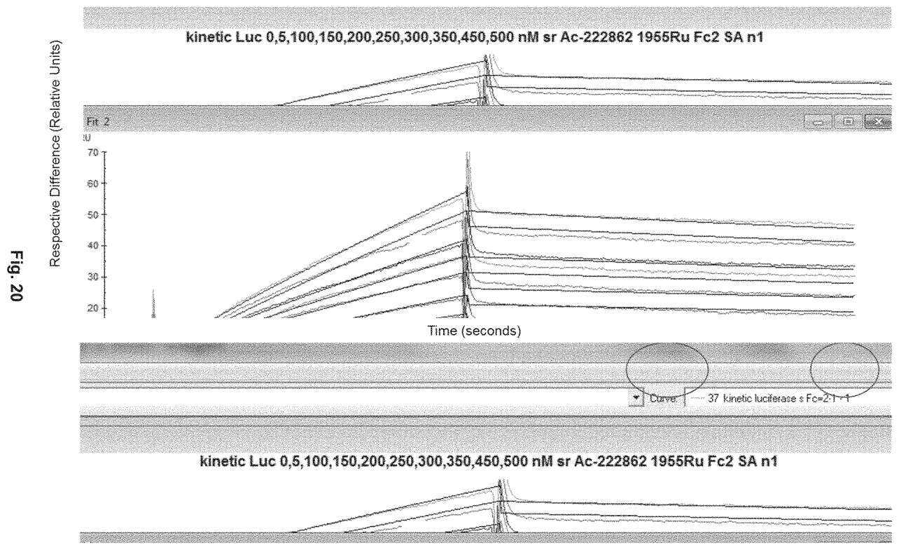

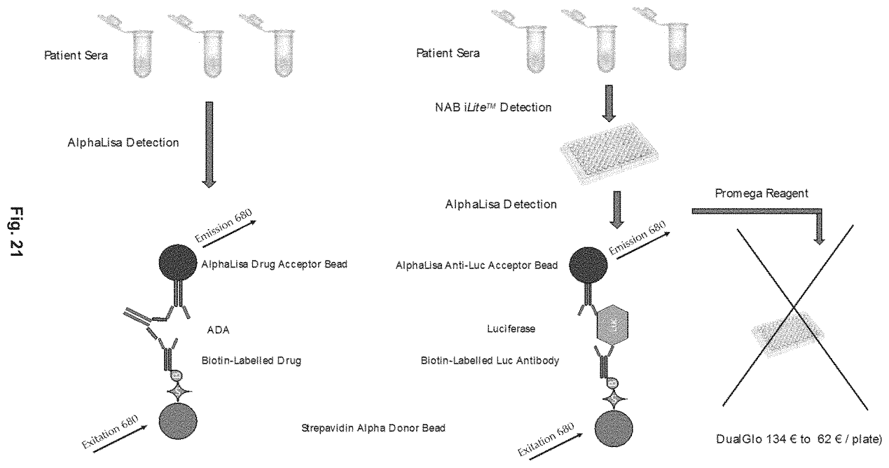

Surface plasmon resonance (SPR) occurs when polarized light strikes an electrically conducting surface at the interface between two materials. This generates electron charge density waves called plasmons, that reduce the intensity of reflected light at a specific angle known as the resonance angle, in proportion to the mass on a sensor surface. This angle changes as molecules bind and dissociate and the interaction profile is thus recorded in real time in a sensorgram. SPR allows real-time, label-free detection of biomolecular interactions including protein-protein interactions such as those between an antibody and a protein antigen. In an initial experiment designed to determine the specificity of the interaction, a biotin labelled antibody directed against the V5 protein of Simian virus 5 (Ab-18617, AbCam, UK) was bound to a streptavidin coated Biacore 3000 sensor chip. No significant increase in signal relative to the background reading was observed when recombinant firefly luciferase was applied to the sensor chip coated with the Anti-V5 antibody (FIG. 17A). In contrast, recombinant firefly luciferase was readily detected when applied to the sensor chip using a biotin labelled monoclonal anti-luciferase antibody Ab-181640 (AbCam UK) bound to a streptavidin coated Biacore 3000 sensor chip (FIGS. 17B and 18). A clear dose related increase in signal was also observed when increasing concentrations of recombinant firefly luciferase were applied to the sensor chip to which a biotin labelled monoclonal anti-luciferase antibody Ab-181640 (AbCam UK) was bound to a streptavidin coated Biacore 3000 sensor chip (FIGS. 19 and 20). SPR allows the binding affinity and dissociation constants (Kd) of the interaction of two molecules to be determined. The Kd of the capture antibody Ab-181640 (AbCam UK) for recombinant firefly luciferase using the Biacore 3000 was found to be 3.4·10⁻⁷ (FIG. 20).

### Example 5

The PerkinElmer AlphaLISA solution ELISA platform technology is based on the use of streptavidin coated donor beads and a detection system based on digoxigenin-labelled acceptor beads in which an ADA forms a bridge between a biotin labelled drug attached to the streptavidin coated donor beads and the digoxigenin-labelled acceptor beads. The labelled drug ADA complexes are detected using anti-digoxigenin-HRP conjugate and quantification of the luminescence signal (FIG. 3). A different dual labelled drug pair is required for each AlphaLISA ADA assay. In contrast, a single pair of anti-luciferase antibody acceptor beads and biotin labelled donor beads can be used for the quantification of the potency, NAb response, and effector cell response for all the drugs tested using the invention that allows cell-based assays to be quantified using the AlphaLISA platform.

In one aspect, the invention relates to the following items:

1. A kit comprising a cell, buffer containing a detergent or passive lysis buffer suitable for lysing cells, an anti-luciferase antibody one part labelled with a fluorescent tag and the other part with a second tag such as biotin, wherein the anti-luciferase antibody comprises one or more of the following features in any combination(s);
the anti-luciferase antibody is a monoclonal antibody,
the anti-luciferase antibody is a polyclonal antibody,
the anti-luciferase antibody is specific for firefly luciferase,
the anti-luciferase antibody is specific for Renilla luciferase,
the anti-luciferase antibody is specific for Nano luciferase,
the anti-luciferase antibody is specific for Gaussia luciferase,
the anti-luciferase antibody is labelled with a fluorescent tag such as e.g. Alexa,
the anti-luciferase antibody is labelled with a Sulfo-Tag,
the anti-luciferase antibody is labelled with biotin,
the anti-luciferase antibody is labelled with horseradish peroxidase (HRP),
the anti-luciferase antibody is labelled with digoxigenin (DIG)
the anti-luciferase antibody is recognized by a secondary antibody,
the anti-luciferase antibody is attached to streptavidin coated donor beads,
the anti-luciferase antibody is attached to digoxigenin-labelled acceptor beads.

2. The cell according to item 1, wherein the cell expresses a first reporter gene product and further expresses a second reporter protein which is different from the first reporter protein, and wherein said cell comprises one or more of the following features in any combination(s);
the cell expresses a reporter gene under the control of a drug specific promoter,
the cell expresses a second reporter protein, which is different from the first reporter protein, under the control of a constitutive promoter,
the cell expresses a luciferase reporter gene under the control of a drug specific promoter,
the cell expresses a luciferase reporter gene encoding, an anthozoan luciferase protein or a decapod crustacean luciferase protein, under the control of a drug specific promoter,
the cell expresses firefly luciferase reporter gene under the control of a drug specific promoter,
the cell expresses Renilla luciferase reporter gene under the control of a drug specific promoter,
the cell expresses Nano luciferase reporter gene under the control of a drug specific promoter,
the cell expresses Gaussia luciferase reporter gene under the control of a drug specific promoter,
the cell expresses a second reporter protein, which is different from the first reporter protein, under the control of a constitutive promoter,
the cell expresses a luciferase reporter gene encoding, an anthozoan luciferase protein or a decapod crustacean luciferase protein, under the control of a constitutive promoter,
the cell expresses a luciferase reporter gene under the control of a constitutive promoter,
the cell expresses firefly luciferase reporter gene under the control of a constitutive promoter,
the cell expresses Renilla luciferase reporter gene under the control of a constitutive promoter,
the cell expresses Nano luciferase reporter gene under the control of a constitutive promoter,
the cell expresses Gaussia luciferase reporter gene under the control of a constitutive promoter.

3. The cell according to any of the preceding items, wherein the cell expresses a reporter gene product operationally linked to CD16a or CD32 and that the reporter gene product responds to ligation of the Fc moiety of an antibody bound to CD16 and a second cell that expresses an antigen recognized by a therapeutic antibody the ADCC activity of which is to be determined, such that interaction of the antibody with the specific antigen on the target cell results in receptor aggregation and activation of the reporter gene on the effector cell.

4. The cell according to any of the preceding items, wherein the cell expresses a reporter gene product operationally linked to CD16a or CD32 and that the reporter gene product responds to ligation of the Fc moiety of an antibody bound to CD16a or CD32 and a second target cell containing a protease cleavable reporter-gene product such as a luciferase that responds to the release of a protease and that once secreted can be quantified allowing the assessment of ADCP activity, or alternatively, the protease may induce a conformational change in the reporter-gene protein product that results in the appearance of a hidden epitope that can be detected by the antibody pair used in the platform detection system.

5. The cell according to any of the preceding items, wherein the cell constitutes a target cell containing a protease cleavable reporter-gene product such as a luciferase the activity of which is either increased or inhibited by the release of a protease and that once secreted, or released spontaneously or released by the lysis of the cells can be quantified allowing the assessment of CDC activity, or alternatively, the protease may induce a conformational change in the reporter-gene protein product that results in the appearance of a hidden epitope that can be detected by the antibody pair used in the platform detection system.

6. The cell according to any of the preceding items for use in the quantification of the potency of cells used in adoptive therapy such as CAR-T cells, wherein the cell constitutes a target cell containing a protease cleavable reporter-gene product such as a luciferase that responds to the release of a protease that once secreted can be quantified allowing the assessment of cytotoxicity, or alternatively, the protease induces a conformational change in the reporter-gene protein product that results in the appearance of a hidden epitope that can be detected by the antibody pair used in the platform detection system.

7. A kit or kit of parts, comprising:

i) a cell according to any of items 1-6;

ii) a cell in which the endogenous target to which an antibody is specific is invalidated/mutated,

iii) a cell in which the expression of the target to which an antibody is specific is enhanced.

8. The kit according to item 7, wherein the target that is CD19, CD20, mTNFalpha, erbB2, EGFR.

9. The kit according to any of items 7-8, wherein the kit comprises two vials and wherein the cells in i) and iii) are present in one and the same vial at the optimal E:T ratio.

10. The kit according to items 9, wherein the ratio between the cell in i) and the target cell in iii) (E:T ratio) is in range from about 24:1 to about 2:1, or e.g. about 6:1, or about e.g. 3:1, or about e.g. 1.5:1.

11. A method for quantifying the ADCC activity ex vivo in clinical samples from patients treated with therapeutic antibodies, the method comprising the steps of;

a) contacting a sample obtained from a patient undergoing treatment comprising administration of an antibody, with target cells ii) in item 7,

b) subtracting the signal obtained in the presence of cells i), in which the drug target has been invalidated, from the signal obtained in the presence of effector cells i) and target cells iii),

c) determining the ADCC activity on the basis of the signal relationship—as measured in a) and b), and wherein a positive result for a serum sample (i.e. detectable ADCC activity) is present when the signal relationship for Effector cells i) & Target ++ cells iii)/Effector cells i) & Target −/− cells ii) and wherein a negative result for a serum sample (i.e. no detectable ADCC activity) is present when the value for Effector cells i) & Target ++ cells iii)/value for Effector cells i) & Target −/− cells ii) 1.

12. A method for compensating for the non-specific increase in the reporter-gene signal in the presence of human serum, the method comprising: subtracting the signal obtained in the presence of cells i), from the signal obtained in the presence of effector cells i) and target cells iii) in item 7, that express the drug target or serum samples that exhibit activity that is not related to ADCC activity specific to the antibody under investigation when Effector cells i) and target negative cells ii) >1.

13. A method for conducting a cell based assay comprising the steps of;

i) carrying out a cell based reporter gene assay in the usual manner except that on completion of the assay luciferase activity is either secreted or released by lysis of the cells a suitable means such as e.g. the addition of a detergent or a passive lysis buffer,

ii) followed by addition of an antibody, that specifically recognizes the reporter gene product, a proportion of which is labelled with one of the two tags specific for an ELISA or a particular automated immuno-assay platform and the other proportion of which is labelled with the second tag specific for the ELISA or the same automated immuno-assay platform,

iii) adding an appropriate volume of the supernatant of the cell lysate to the specific automated immuno-assay platform and quantified in the usual manner, or alternatively the drug-specific effects may also be normalized by the addition of a second antibody specific for a second reporter gene product different from the first and transcribed under the control of a constitutive promoter.

In a further aspect, the invention also relates to the following articles:

1. A kit comprising a cell, buffer containing a detergent or passive lysis buffer suitable for lysing cells, an anti-luciferase antibody one part labelled with a fluorescent tag and the other part with biotin with one or more of the below options:
wherein the anti-luciferase antibody is a monoclonal antibody, or
wherein the anti-luciferase antibody is a polyclonal antibody, and/or
wherein the anti-luciferase antibody is specific for firefly luciferase, and/or
wherein the anti-luciferase antibody is specific for Renilla luciferase, and/or
wherein the anti-luciferase antibody is specific for Nano luciferase, and/or
wherein the anti-luciferase antibody is specific for Gaussia luciferase, and/or
wherein the anti-luciferase antibody is labelled with a fluorescent tag such as Alexa, and/or
wherein the anti-luciferase antibody is labelled with a Sulfo-Tag, and/or
wherein the anti-luciferase antibody is labelled with biotin, and/or
wherein the anti-luciferase antibody is attached to streptavidin coated donor beads, and/or
wherein the anti-luciferase antibody is attached to digoxigenin-labelled acceptor beads, and/or
wherein the anti-luciferase antibody is detected using a secondary antibody that is labelled, with biotin, Alexa, a Sulfo-Tag, digoxigenin or other label that permits either attachment or detection of the antibody.

2. The cell according to any of the preceding articles, wherein the cell expresses a first reporter gene product and further expresses a second reporter protein which is different from the first reporter protein with one or more of the below options:
wherein the cell expresses a reporter gene under the control of a drug specific promoter, and/or
wherein the cell expresses a second reporter protein, which is different from the first reporter protein, under the control of a constitutive promoter, and/or
wherein the cell expresses a luciferase reporter gene encoding, an anthozoan luciferase protein or a decapod crustacean luciferase protein, under the control of a drug specific promoter, and/or
wherein the cell expresses a luciferase reporter gene under the control of a drug specific promoter, and/or
wherein the cell expresses firefly luciferase reporter gene under the control of a drug specific promoter, and/or
wherein the cell expresses Renilla luciferase reporter gene under the control of a drug specific promoter, and/or
wherein the cell expresses Nano luciferase reporter gene under the control of a drug specific promoter, and/or
wherein the cell expresses Gaussia luciferase reporter gene under the control of a drug specific promoter, and/or
wherein the cell expresses a second reporter protein, which is different from the first reporter protein, under the control of a constitutive promoter, and/or
wherein the cell expresses a luciferase reporter gene encoding, an anthozoan luciferase protein or a decapod crustacean luciferase protein, under the control of a constitutive promoter, and/or
wherein the cell expresses a luciferase reporter gene under the control of a constitutive promoter, and/or
wherein the cell expresses firefly luciferase reporter gene under the control of a constitutive promoter, and/or
wherein the cell expresses Renilla luciferase reporter gene under the control of a constitutive promoter, and/or
wherein the cell expresses Nano luciferase reporter gene under the control of a constitutive promoter, and/or
wherein the cell expresses Gaussia luciferase reporter gene under the control of a constitutive promoter.

3. The cell according to any of the preceding articles, wherein the cell expresses a reporter gene product operationally linked to CD16a and that the reporter gene product responds to ligation of the Fc moiety of an antibody bound to CD16 and a second cell that expresses an antigen recognized by a therapeutic antibody the ADCC activity of which is to be determined. The interaction of the antibody with the specific antigen on the target cell results in receptor aggregation and activation of the reporter gene on the effector cell.

4. The cell according to any of the preceding articles, wherein the cell expresses a reporter gene product operationally linked to CD32 and that the reporter gene product responds to ligation of the Fc moiety of an antibody bound to CD32 and a second target cell containing a protease cleavable reporter-gene product such as a luciferase that responds to the release of a protease and that once secreted can be quantified allowing the assessment of ADCP activity. Alternatively, the protease may induce a conformational change in the reporter-gene protein product that results in the appearance of a hidden epitope that can be detected by the antibody pair used in the platform detection system.

5. The cell according to any of the preceding articles, wherein the cell constitutes a target cell containing a protease cleavable reporter-gene product such as a luciferase that responds to the release of a protease and that once secreted can be quantified allowing the assessment of CDC activity. Alternatively, the protease may induce a conformational change in the reporter-gene protein product that results in the appearance of a hidden epitope that can be detected by the antibody pair used in the platform detection system.

6. The cell according to any of the preceding articles for the quantification of the potency of cells used in adoptive therapy such as CAR-T cells, wherein the cell constitutes a target cell containing a protease cleavable reporter-gene product such as a luciferase that responds to the release of a protease that once secreted can be quantified allowing the assessment of cytotoxicity, or alternatively, the protease may induce a conformational change in the reporter-gene protein product that results in the appearance of a hidden epitope that can be detected by the antibody pair used in the platform detection system.

7. The cell according to any of the preceding articles, wherein the cell expresses a reporter gene product operationally linked to TLR2 or TLR9 and that the reporter gene product responds to infection of the cell with either Adeno associated Virus (AAV) or a recombinant AAV virus expressing a transgene for use in gene therapy. Such a cell can be used to quantify the potency of an AAV vector or the neutralizing anti-AAV antibody response to either wild type AAV or to a recombinant AAV vector since the presence of anti-AAV neutralizing antibodies in contact with AAV will inhibit its ability to activate the TLR2 or TLR9 responsive reporter gene product.

8. A kit, comprising: i) a cell according to any of the preceeding articles; ii) a cell in which the endogenous target to which an antibody is specific is invalidated (mutated); and iii) a cell in which the expression of the target to which an antibody is specific is enhanced.

9. The kit according to article 8, wherein the target that is CD20, mTNFalpha, erbB2, EGFR.

10. The kit according to any of the preceding articles, wherein the kit comprises two vials and wherein the cells in i) and iii) are present in one and the same vial at the optimal E:T ratio.

11. The kit according to any of the preceding articles, wherein the ratio between the cell in i) and the target cell in iii) (E:T ratio) is in range from about 24:1 to about 2:1, or e.g. about 6:1, or about e.g. 3:1, or about e.g. 1.5:1.

12. The kit according to any of the preceding articles, wherein the cells in i) and iii) are present in one and the same vial at the optimal E:T ratio together with optimum concentrations of the capture and detection anti-luciferase antibodies.

13. A method for quantifying the ADCC activity ex vivo in clinical samples from patients treated with therapeutic antibodies, the method comprising the steps of;

a) contacting a sample obtained from a patient undergoing treatment comprising administration of an antibody, with target cells ii) in the preceding articles,

b) subtracting the signal obtained in the presence of cells i), in which the drug target has been invalidated, from the signal obtained in the presence of effector cells i) and target cells iii),

c) determining the ADCC activity on the basis of the signal relationship—as measured in a) and b), and wherein a positive result for a serum sample (i.e. detectable ADCC activity) is present when the signal relationship for Effector cells i) & Target ++ cells iii)/Effector cells i) & Target −/− cells ii) and wherein a negative result for a serum sample (i.e. no detectable ADCC activity) is present when the value for Effector cells i) & Target ++ cells iii)/value for Effector cells i) & Target −/− cells ii) 1.

14. A method for quantifying the neutralizing activity ex vivo in clinical samples from patients prior to treatment with recombinant AAV vectors to determine their neutralizing antibodies titers against different AAV serotypes, or during treatment with recombinant AAV vectors to determine their neutralizing antibodies titers against the virus vector and/or the transgene carried by the virus vector with the method comprising the steps of;

a) contacting a sample obtained from a patient prior to treatment or undergoing treatment comprising administration of an antibody, with reporter cells and AAV or the AAV vector ii) in the preceding articles,
b) subtracting the signal obtained in the presence of cells containing the same reporter gene but without AAV i),
c) determining the neutralizing activity on the basis of the signal relationship—as measured in a) and b), and wherein a positive result for a serum sample (i.e. detectable neutralizing activity) is present when the signal relationship is ≥1 and wherein a negative result for a serum sample (i.e. no detectable neutralizing activity) is present when the value is 1.

15. A method for compensating for the non-specific increase in the reporter-gene signal in the presence of human serum, the method comprising: subtracting the signal obtained in the presence of cells i), from the signal obtained in the presence of effector cells i) and target cells iii) in the preceding articles, that express the drug target or serum samples that exhibit activity that is not related to ADCC activity specific to the antibody under investigation when Effector cells i) & Target negative cells ii) >1.

16. A method for conducting a cell based assay comprising carrying out a cell based reporter gene assay in the usual manner except that on completion of the assay the cells are lysed using a suitable means such as the addition of a detergent or a passive lysis buffer, followed by addition of an antibody, that specifically recognizes the reporter gene product, a proportion of which is labelled with one of the two tags specific for a particular automated immuno-assay platform and the other proportion of which is labelled with the second tag specific for the same automated immuno-assay platform, and wherein an appropriate volume of the supernatant of the cell lysate is then added to the specific automated immuno-assay platform and quantified in the usual manner, and wherein optionally, the drug-specific effects may also be normalized by the addition of a second antibody specific for a second reporter gene product different from the first and transcribed under the control of a constitutive promoter.

### REFERENCES

1. Tovey, M. G., In Detection and Quantification of antibodies to biopharmaceuticals: Practical and Applied Considerations. Editor, Michael G Tovey, John Wiley & Sons Inc; New York. pp 1-11, 2011.
2. Casadevall, et al N. Eng. J. Med. 346:469-475, 2002
3. Li J. et al Blood, 98:3241-3248, 2001
4. Chung C. H. et al Eng. J. Med. 358:1109-1117, 2008
5. Philips, J. T. Arch. Neurol. 64:386-387, 2010
6. Food and Drugs Administration (FDA) Draft guidance for industry: assay development for immunogenicity assays. www.fda.gov/UCM192750. 2009
7. European Medicines Agency (EMA) Immunogenicity assessment of biotechnology derived therapeutic proteins. www.ema.europa.eu/1432706EN.2007
8. F. Marcucci, M. Bellone, C. Rumio, and A. Corti, “Approaches to improve tumor accumulation and interactions between monoclonal antibodies and immune cells.,” MAbs, vol. 5, pp. 34-46, 2013.
9. N. Rajasekaran, C. Chester, A. Yonezawa, X. Zhao, and H. E. Kohrt, “Enhancement of antibody-dependent cell mediated cytotoxicity: a new era in cancer treatment,” Immunotargets Ther, vol. 4, pp. 91-100, 2015.

1. A cell or cell line wherein the cell expresses a first reporter gene product and further expresses a second reporter protein which is different from the first reporter protein, wherein the cell comprises a vector construct further comprising a nucleotide sequence selected from one or more of SEQ ID NO.: 1 to SEQ ID NO.: 4.

2. The cell or cell line according to claim 1, wherein the cell expresses a reporter gene under the control of a drug specific promoter.

3. The cell or cell line according to claim 1, wherein the cell expresses a second reporter protein, which is different from the first reporter protein, under the control of a constitutive promoter.

4. The cell or cell line according to claim 1, wherein the cell expresses a luciferase reporter gene encoding an anthozoan luciferase protein or a decapod crustacean luciferase protein, under the control of a drug specific promoter.

5. The cell or cell line according to claim 1, wherein the cell expresses a luciferase reporter gene under the control of a drug specific promoter.

6. The cell or cell line according to claim 1, wherein the cell expresses firefly luciferase reporter gene under the control of a drug specific promoter.

7. The cell or cell line according to claim 1, wherein the cell expresses a Renilla luciferase reporter gene under the control of a drug specific promoter.

8. The cell or cell line according to claim 1, wherein the cell expresses Nano luciferase reporter gene under the control of a drug specific promoter.

9. The cell or cell line according to claim 1, wherein the cell expresses Gaussia luciferase reporter gene under the control of a drug specific promoter.

10. The cell or cell line according to claim 1, wherein the cell expresses a second reporter protein, which is different from the first reporter protein, under the control of a constitutive promoter.

11. The cell or cell line according to claim 1, wherein the cell expresses a luciferase reporter gene encoding an anthozoan luciferase protein or a decapod crustacean luciferase protein, under the control of a constitutive promoter.

12. The cell or cell line according to claim 1, wherein the cell expresses a luciferase reporter gene under the control of a constitutive promoter.

13. The cell or cell line according to claim 1, wherein the cell expresses a firefly luciferase reporter gene under the control of a constitutive promoter.

14. The cell or cell line according to claim 1, wherein the cell expresses a Renilla luciferase reporter gene under the control of a constitutive promoter.

15. The cell or cell line according to claim 1, wherein the cell expresses a Nano luciferase reporter gene under the control of a constitutive promoter.

16. The cell or cell line according to claim 1, wherein the cell expresses a Gaussia luciferase reporter gene under the control of a constitutive promoter.

17. The cell or cell line according to claim 1, wherein the cell is a mammalian or avian cell.

18. The cell or cell line according to claim 1, wherein the cell is human.

19. The cell or cell line according to claim 1, wherein the cell is one or more of Jurkat, Molt4, Raji, SKBR3, NK92, KHYG-1, HEK293 cells DT-40, PIL-5, or MSB-1.

20. The cell or cell line according to claim 1, wherein the vector construct further comprises a polynucleotide encoding one or more selected from co-stimulatory molecule CD28, co-stimulatory molecule CD137 (4-1BB), co-stimulatory molecule CD247 (T3 Zeta chain), co-stimulatory molecule CD278 (ICOS), or wherein co-stimulatory molecules are receptors selected from one or more of CD28, CD137L (4-1BB), and ICOS.

21. The cell or cell line according to claim 1, wherein the cell expresses a reporter gene product operationally linked to CD16a and wherein the reporter gene product responds to ligation of the Fc moiety of an antibody bound to CD16 and a second cell that expresses an antigen recognized by a therapeutic antibody the ADCC activity of which is to be determined, and wherein the interaction of the antibody with the specific antigen on the target cell results in receptor aggregation and activation of the reporter gene on the effector cell.

22. The cell or cell line according to claim 1, wherein the cell expresses a reporter gene product operably linked to CD32 and that the reporter gene product responds to ligation of the Fc moiety of an antibody bound to CD32 and a second target cell containing a protease cleavable reporter-gene product such as e.g. a luciferase that responds to the release of a protease and that once secreted can be quantified allowing the assessment of ADCP activity, or alternatively, the protease may induce a conformational change in the reporter-gene protein product that results in the appearance of a hidden epitope that can be detected by the antibody pair used in the platform detection system.

23. The cell according to claim 1, wherein the cell expresses a reporter gene product operationally linked to TLR2 or TLR9 and that the reporter gene product responds to infection of the cell with either Adeno associated Virus (AAV) or a recombinant AAV virus expressing a transgene for use in gene therapy.

24. An automated cell-based assay, the improvement wherein the cell or cell line according to claim 1 expresses a reporter protein that is detected and quantified in the automated cell-based assay, such as e.g. automated immunoassay.

25. The automated cell-based assay according to claim 24, wherein the automated assay is any ELISA type of assay or assay platform such as e.g. Gyros, MSD, or AlphaLISA systems, or label-free detection using SPR such as e.g. the Biacore system.

26. A kit or a kit of parts comprising:
i) a cell according to claim 1;
ii) a cell in which the endogenous target to which an antibody is specific is invalidated (mutated); and
iii) a cell in which the expression of the target to which an antibody is specific is enhanced.

27. The kit according to claim 26, wherein the target that is CD20, mTNFalpha, erbB2, EGFR.

28. The kit according to claim 26, wherein the kit comprises two vials and wherein the cells in i) and iii) are present in one and the same vial at the optimal E:T ratio.

29. The kit according to claim 26, wherein the ratio between the cell in i) and the target cell in iii) (E:T ratio) is in range from about 24:1 to about 2:1, or e.g. about 6:1, or about e.g. 3:1, or about e.g. 1.5:1.

30. The kit according to claim 26, wherein the cells in i) and iii) are present in one and the same vial at the optimal E:T ratio together with optimum concentrations of the capture and detection anti-luciferase antibodies.

31-32. (canceled)
